(19) United States
(12) Patent Application Publication
(10) Pub. No.: US 2022/0370627 A1
(43) Pub. Date: Nov. 24, 2022

(54) COMPOSITIONS AND METHODS FOR TARGETED PROTEIN STABILIZATION BY REDIRECTING ENDOGENOUS DEUBIQUITINASES

(71) Applicant: THE TRUSTEES OF COLUMBIA UNIVERSITY IN THE CITY OF NEW YORK, New York, NY (US)

(72) Inventors: Scott KANNER, New York, NY (US); Henry M. COLECRAFT, New York, NY (US)

(21) Appl. No.: 17/864,382

(22) Filed: Jul. 13, 2022

Related U.S. Application Data
(63) Continuation of application No. PCT/US2021/013382, filed on Jan. 14, 2021.
(60) Provisional application No. 62/961,082, filed on Jan. 14, 2020.

(51) Int. Cl. A61K 47/55; A61K 31/404; C12Q 1/6869; C12N 15/10

(52) U.S. Cl. CPC A61K 47/55; A61K 31/404; C12Q 1/6869; C12N 15/1093

(57) ABSTRACT

The present disclosure provides, inter alia, bivalent small molecules and methods for treating or ameliorating the effects of a disease, such as long QT syndrome, or cystic fibrosis, in a subject, using the bivalent small molecules disclosed herein. Also provided are methods of identifying and preparing small molecule binders that target proteins of interest.

### CROSS REFERENCE TO RELATED APPLICATIONS

The present application is a continuation application of PCT International Application No. PCT/US2021/013382, filed Jan. 14, 2021, which claims benefit of U.S. Provisional Patent Application Ser. No. 62/961,082, filed on Jan. 14, 2020, which applications are incorporated by reference herein in their entireties.

### GOVERNMENT FUNDING

This invention was made with government support under grant no. HL122421, awarded by the National Institutes of Health. The government has certain rights in the invention.

### FIELD OF THE DISCLOSURE

The present disclosure provides, inter alia, bivalent small molecules and methods for treating or ameliorating the effects of a disease, such as long QT syndrome, or cystic fibrosis, in a subject, using such bivalent molecules.

### BACKGROUND OF THE DISCLOSURE

Protein stability is critical for the proper function of all proteins in the cell. Many disease processes stem from deficits in the stability or expression of one or more proteins, ranging from inherited mutations that destabilize ion channels (i.e. cystic fibrosis, CFTR), to viral-mediated elimination of host defenses (i.e. MHCI receptors) and degradation of cell cycle inhibitors in tumor cell proliferation (i.e. p27, p21). Ubiquitin is a key post-translational modification that is a master regulator of protein turnover and degradation. Nevertheless, the widespread biological role and promiscuity of ubiquitin signaling has provided a significant barrier in developing therapeutics that target this pathway to selectively stabilize a given protein-of-interest.

Ubiquitination is mediated by a step-wise cascade of three enzymes (E1, E2, E3), resulting in the covalent attachment of the 76-residue ubiquitin to exposed lysines of a target protein. Ubiquitin itself contains seven lysines (K6, K11, K27, K29, K33, K48, K63) that, together with its N-terminus (Met1), can serve as secondary attachment points, resulting in a diversity of polymeric chains, differentially interpreted as sorting, trafficking, or degradative signals. Ubiquitination has been associated with inherited disorders (cystic fibrosis, cardiac arrhythmias, epilepsy, and neuropathic pain), metabolic regulation (cholesterol homeostasis), infectious disease (hijacking of host system by viral and bacterial pathogens), and cancer biology (degradation of tumor suppressors, evasion of immune surveillance).

Deubiquitinases (DUBs) are specialized isopeptidases that provide salience to ubiquitin signaling through the revision and removal of ubiquitin chains. There are over 100 human DUBs, comprising 6 distinct families: 1) the ubiquitin specific proteases (USP) family, 2) the ovarian tumor proteases (OTU) family, 3) the ubiquitin C-terminal hydrolases (UCH) family, 4) the Josephin domain family (Josephin), 5) the motif interacting with ubiquitin-containing novel DUB family (MINDY), and 6) the JAB1/MPN/Mov34 metalloenzyme domain family (JAMM). Each class of DUBs have their own distinct catalytic properties, with the USP family hydrolyzing all ubiquitin chain types, in stark contrast to the JAMM and OTU families, which contains a diverse set of enzymes with distinct ubiquitin linkage preferences. Recently, DUBs have garnered interest as drug targets, with multiple companies pursuing DUB inhibitors. However, targeting DUBs for therapy has challenges, owing to promiscuity in DUB regulation pathways wherein individual DUBs typically target multiple protein substrates, and particular substrates can be regulated by multiple DUB types.

Ion channelopathies characterized by abnormal trafficking, stability, and dysfunction of ion channels/receptors constitute a significant unmet clinical need in human disease. Inherited ion channelopathies are rare diseases that encompass a broad range of disorders in the nervous system (epilepsy, migraine, neuropathic pain), cardiovascular system (long QT syndrome, Brugada syndrome), respiratory (cystic fibrosis), endocrine (diabetes, hyperinsulinemic hypoglycemia), and urinary (Bartter syndrome, diabetes insipidus) system. Although next generation genomic sequencing has revealed a rapidly expanding list of thousands of channel mutations (with diverse underlying mechanisms of pathology), these rare diseases are almost exclusively treated symptomatically. For example, cystic fibrosis, the most common lethal genetic disease in Caucasians arises due to defects in the cystic fibrosis transmembrane conductance regulator (CFTR), a chloride ion channel. The most studied mutation (ΔF508), accounts for ˜85% of all cases, and causes channel misfolding and ubiquitin-dependent trafficking defects. In another devastating disease, Long QT Syndrome, over 500 mutations in two channels (KCNQ1, hERG) encompasses nearly 90% of all inherited cases. Trafficking deficits in the two channels is the mechanistic basis for a majority of the disease-causing mutations. As such, understanding the underlying cause of loss-of-function is critical for employing a personalized strategy to treat the underlying functional deficit in each disease.

### SUMMARY OF THE DISCLOSURE

The present disclosure provides a bivalent molecule comprising: a) a deubiquitinase (DUB) binder; b) a target binder; and c) a variable linker between the DUB binder and the target binder, wherein the DUB binder is a small molecule.

The present disclosure also provides a method of treating or ameliorating the effects of a disease in a subject, comprising administering to the subject an effective amount of a bivalent molecule disclosed herein.

The present disclosure also provides a method of identifying and preparing a small molecule binder targeting a protein of interest, comprising: a) generating a DNA-encoded compound library; b) incubating the library with the protein of interest; c) washing off unbound molecules; d) amplifying the oligonucleotide codes of the binding compounds by PCR and constructing an enriched compound library; e) repeating steps b) to d) with the enriched library as necessary to further enrich the library containing the oligonucleotide codes of the binding compounds; and f) identifying the small molecule binders by decoding the library generated in step e) for binding validation.

### BRIEF DESCRIPTION OF THE DRAWINGS

The application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIG. 1A shows the underlying symptoms and current treatments for cystic fibrosis (CF). FIG. 1B is a schematic detailing the ubiquitin-dependent regulation of CFTR surface expression, stability, and function. Forward trafficking pathways highlighted in blue, and reverse trafficking pathways highlighted in red.

In FIG. 2, Left, shows the structure of an exemplary protein target, CFTR. NBD1 highlighted in red. Right, Structure of stabilizing enzyme, DUB. FIG. 3 shows a schematic of the “all small-molecule” ReSTORx.

FIG. 4 shows the identification of small-molecule DUB binders using proprietary DNA-encoded compound library technology.

FIG. 5 shows that potential small-molecule DUB binders are validated using cell-free binding assays.

FIG. 6A shows compound screening using a DNA-encoded library approach. Right, shows binding kinetics validation of an exemplary hit compound obtained with optical interferometry. FIG. 6B shows the validation results of some exemplary small molecules. FIG. 6C shows the chemical template for Halo-Targeted ReSTORx molecules, with an “active” DUB-binding component and a “targeting” HaloTag ligand component. FIG. 6D shows the FRET target engagement assay for lead DUB binders, consisting of Cerulean-tagged DUB and Venus-tagged HaloTag constructs co-expressed in HEK293 cells. FIG. 6E is a schematic for an ubiquitin-dependent stabilization assay. The reporter construct is comprised of a YFP-tagged HaloTag and an uncleavable N-terminal ubiquitin fusion that forces poly-ubiquitination and degradation under basal conditions. DUB-recruitment by an “active” component, results in the removal of poly-ubiquitin chains, stabilization of the reporter, and bright YFP fluorescence. FIG. 6F shows the chemical template for first-generation CF-targeted ReSTORx small-molecules, using lumacaftor as an exemplary “targeting” component. FIG. 6G provides the validation studies for mutant CFTR rescue with lead ReSTORx molecules.

### DETAILED DESCRIPTION OF THE DISCLOSURE

One embodiment of the present disclosure is a bivalent molecule comprising: a) a deubiquitinase (DUB) binder; b) a target binder; and c) a variable linker between the DUB binder and the target binder, wherein the DUB binder is a small molecule.

In some embodiments, the DUB is endogenous. In some embodiments, the DUB is selected from the ubiquitin specific proteases (USP) family, the ovarian tumor proteases (OTU) family, the ubiquitin C-terminal hydrolases (UCH) family, the Josephin domain family (Josephin), the motif interacting with ubiquitin-containing novel DUB family (MINDY), and the JAB1/MPN/Mov34 metalloenzyme domain family (JAMM). In some embodiments, the DUB is USP21 or USP2.

In some embodiments, the small molecule binds to a USP family member. In some embodiments, the small molecule binds to a USP2. In some embodiments, the

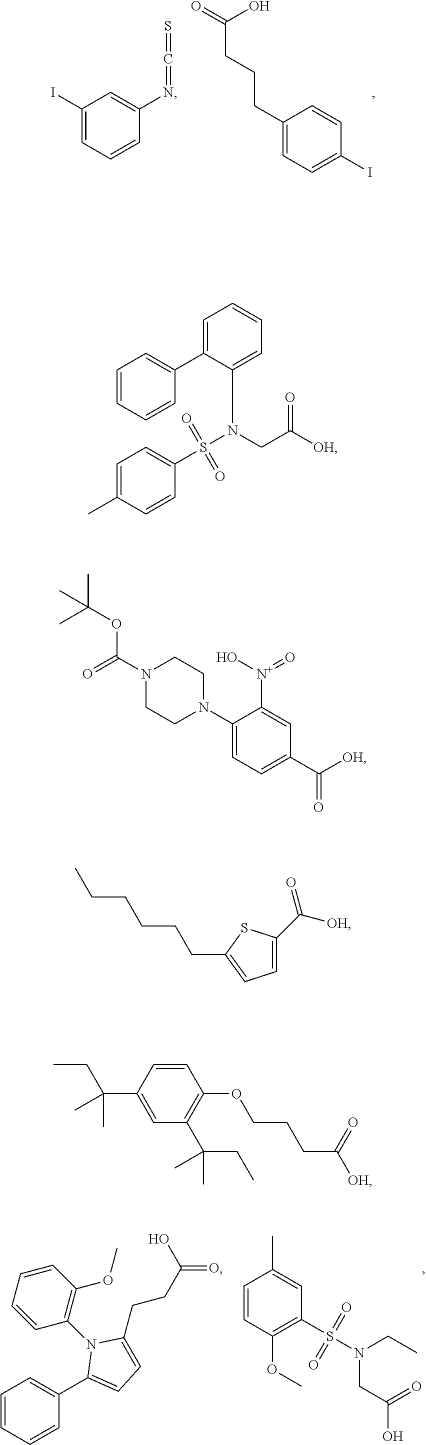

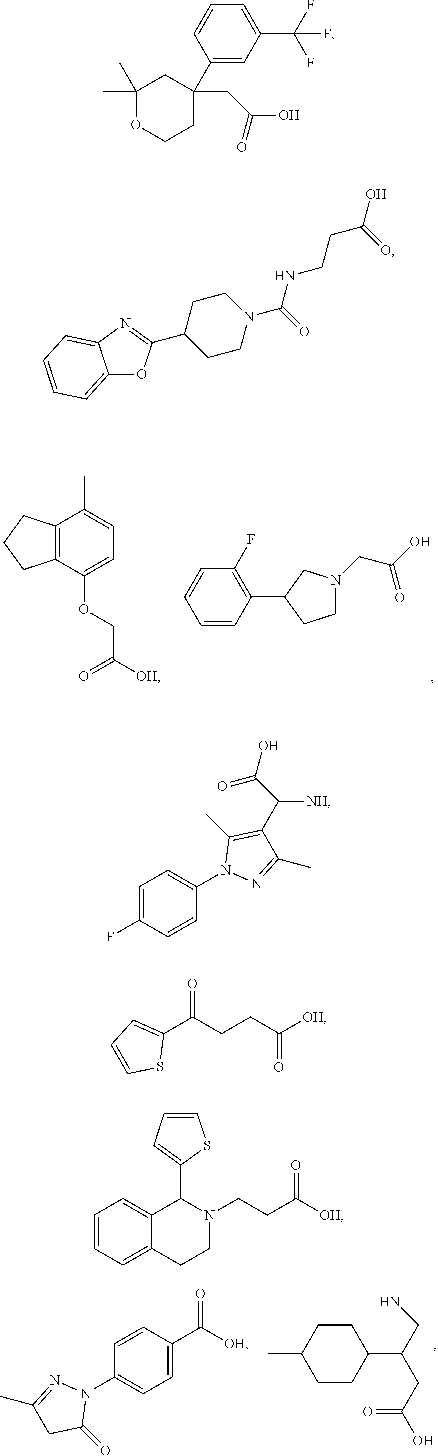

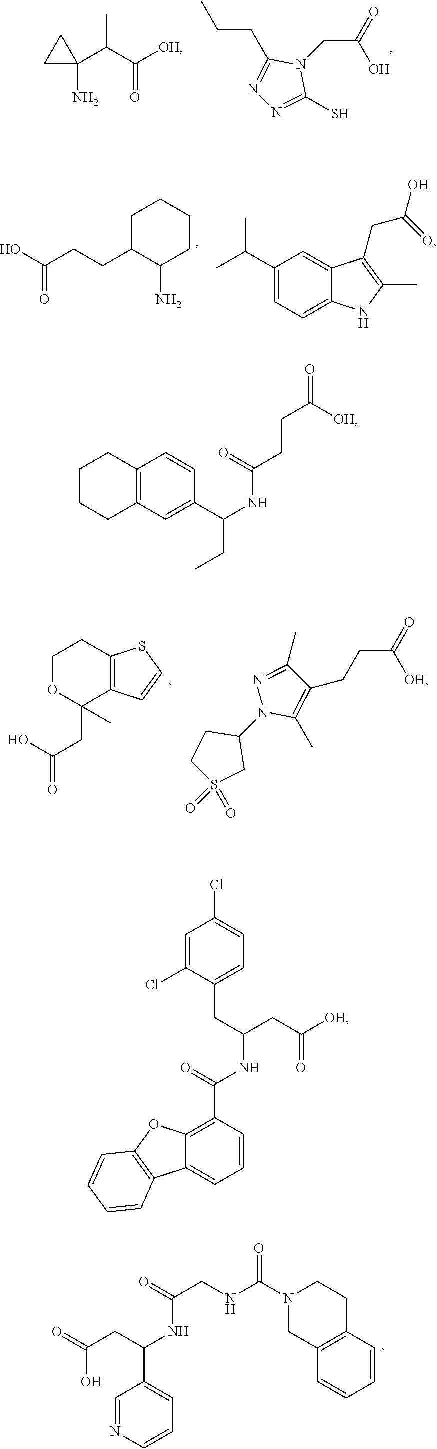

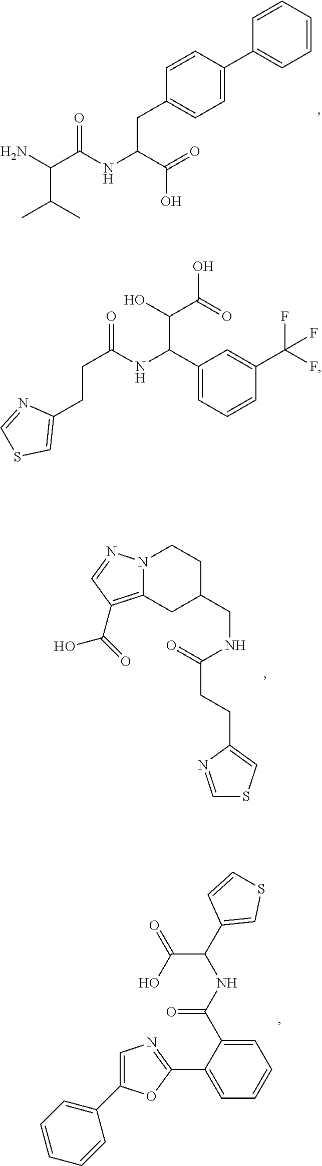 [SMILES: CC(C)C(N)C(=O)NC(Cc1ccc(-c2ccccc2)cc1)C(=O)O ; O=C(CCc1cscn1)NC(c1cccc(C(F)(F)F)c1)C(O)C(=O)O ; O=C(CCc1cscn1)NCC1CCn2ncc(C(=O)O)c2C1 ; O=C(NC(C(=O)O)c1ccsc1)c1ccccc1-c1ncc(-c2ccccc2)o1]

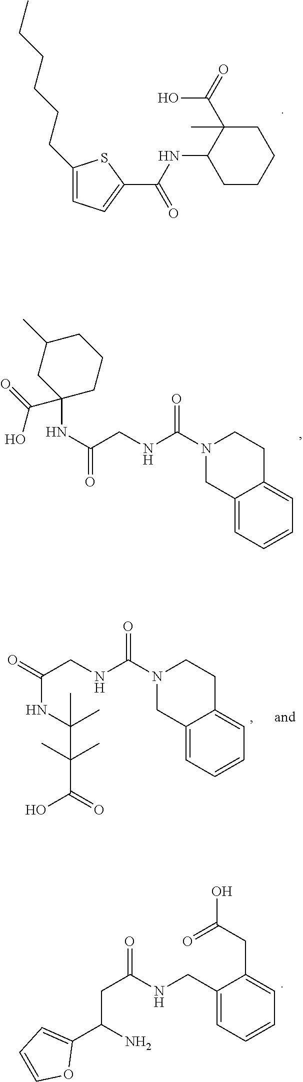 [SMILES: CCCCCCc1ccc(C(=O)NC2CCCCC2(C)C(=O)O)s1 ; CC1CCCC(NC(=O)CNC(=O)N2CCc3ccccc3C2)(C(=O)O)C1 ; CC(C)(NC(=O)CNC(=O)N1CCc2ccccc2C1)C(C)(C)C(=O)O ; NC(CC(=O)NCc1ccccc1CC(=O)O)c1ccco1]

small molecule is selected from:

Preferably, the small molecule is selected from:

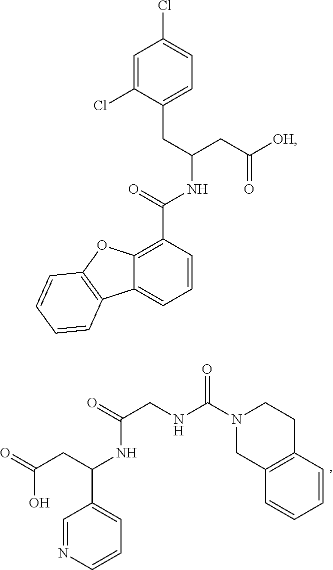 [SMILES: O=C(O)CC(Cc1ccc(Cl)cc1Cl)NC(=O)c1cccc2c1oc1ccccc12 ; O=C(O)CC(NC(=O)CNC(=O)N1CCc2ccccc2C1)c1cccnc1]

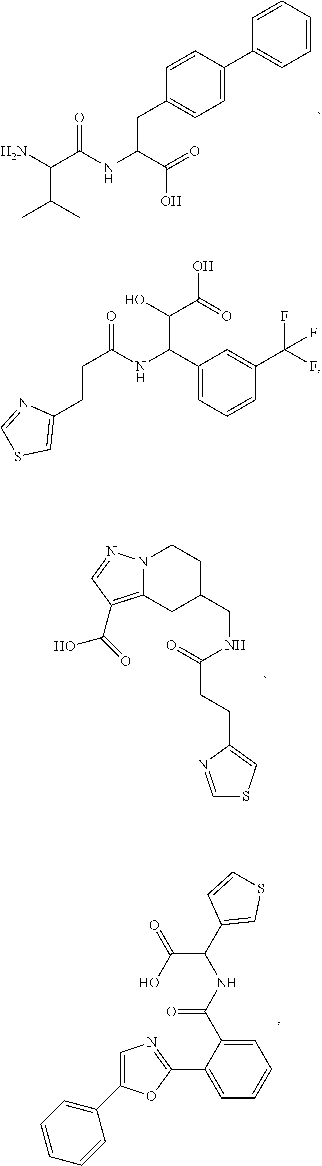 [SMILES: CC(C)C(N)C(=O)NC(Cc1ccc(-c2ccccc2)cc1)C(=O)O ; O=C(CCc1cscn1)NC(c1cccc(C(F)(F)F)c1)C(O)C(=O)O ; O=C(CCc1cscn1)NCC1CCn2ncc(C(=O)O)c2C1 ; O=C(NC(C(=O)O)c1ccsc1)c1ccccc1-c1ncc(-c2ccccc2)o1]

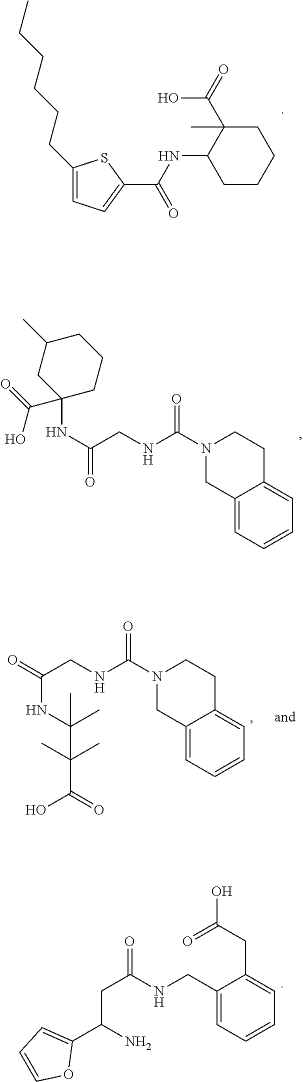 [SMILES: CCCCCCc1ccc(C(=O)NC2CCCCC2(C)C(=O)O)s1 ; CC1CCCC(NC(=O)CNC(=O)N2CCc3ccccc3C2)(C(=O)O)C1 ; CC(C)(NC(=O)CNC(=O)N1CCc2ccccc2C1)C(C)(C)C(=O)O ; NC(CC(=O)NCc1ccccc1CC(=O)O)c1ccco1]

small molecule is selected from:

In some embodiments, aberrant ubiquitination of the target to which the target binder binds causes a disease. In some embodiments, the disease is an inherited ion channelopathy. As used herein, the term “inherited ion channelopathy” refers to rare diseases that encompass a broad range of disorders in the nervous system, cardiovascular system, respiratory system, endocrine system, and urinary system. In the present disclosure, an “inherited ion channelopathy” includes but is not limited to: epilepsy, migraine, neuropathic pain, cardiac arrhythmias, long QT syndrome, Brugada syndrome, cystic fibrosis, diabetes, hyperinsulinemic hypoglycemia, Bartter syndrome, and diabetes insipidus. In some embodiments, the disease is long QT syndrome. In some embodiments, the disease is cystic fibrosis.

In some embodiments, the target to which the target binder binds is cystic fibrosis transmembrane conductance regulator (CFTR).

In some embodiments, the target binder is a small molecule. In some embodiments, the small molecule binds to NBD1 domain of cystic fibrosis transmembrane conductance regulator (CFTR). In some embodiments, the small molecule is selected from:

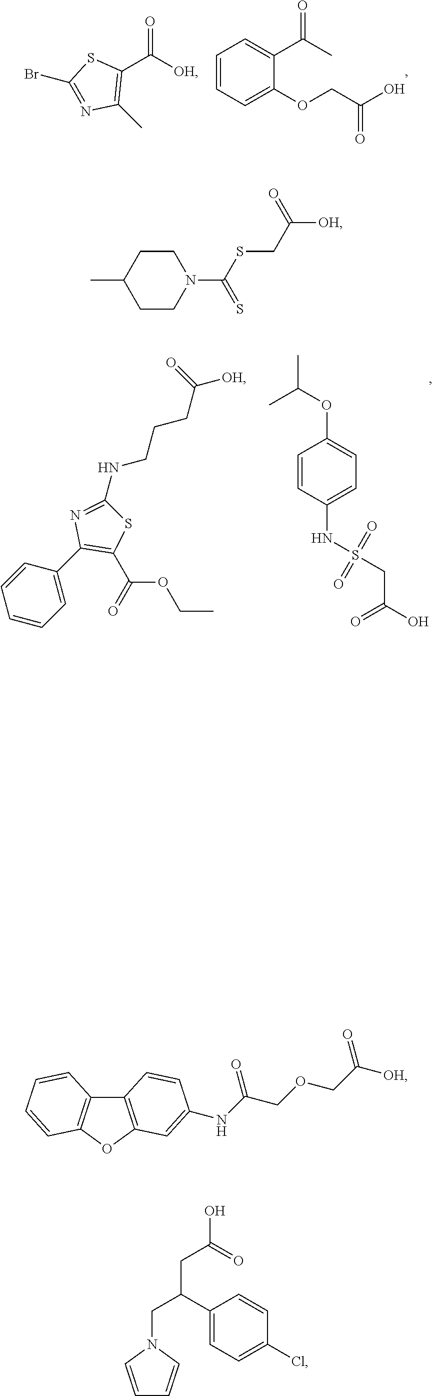

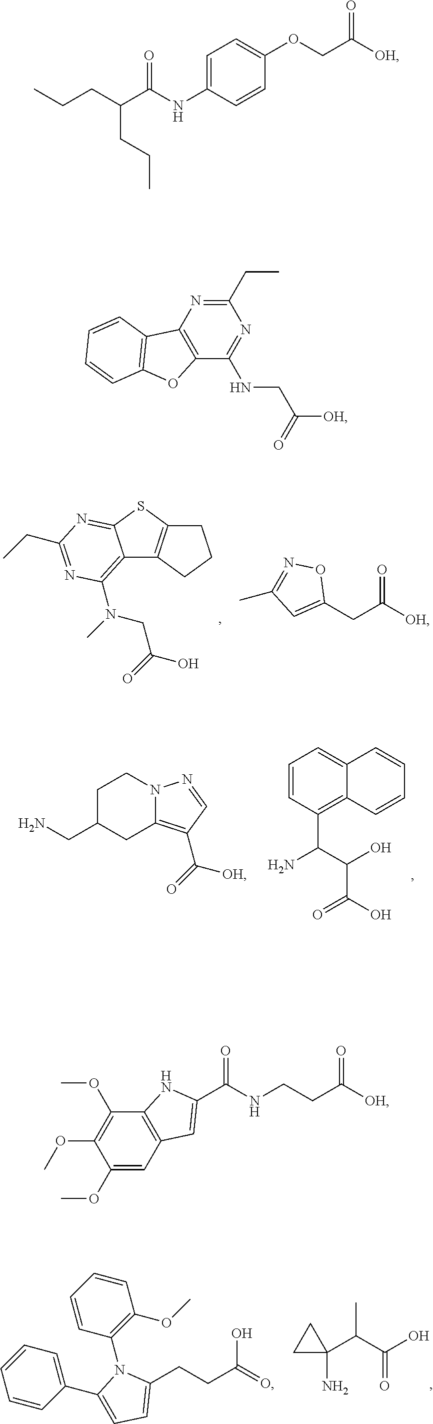

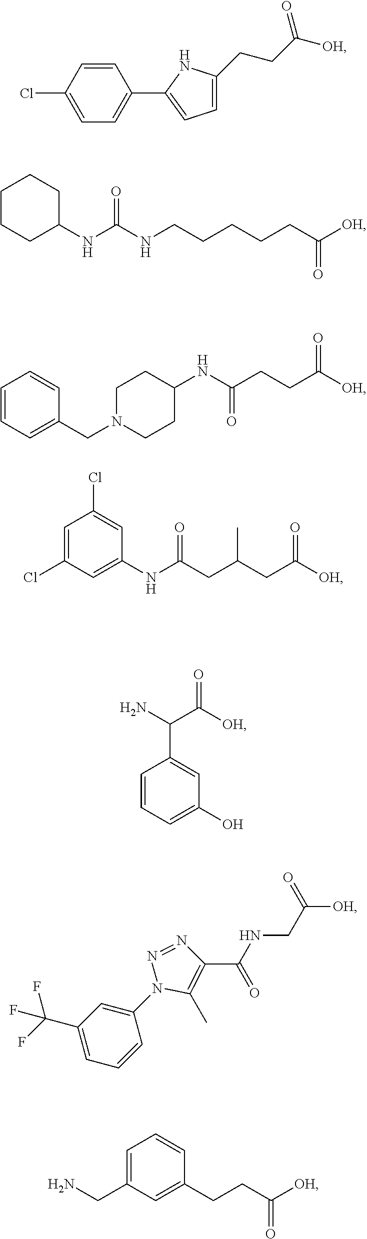

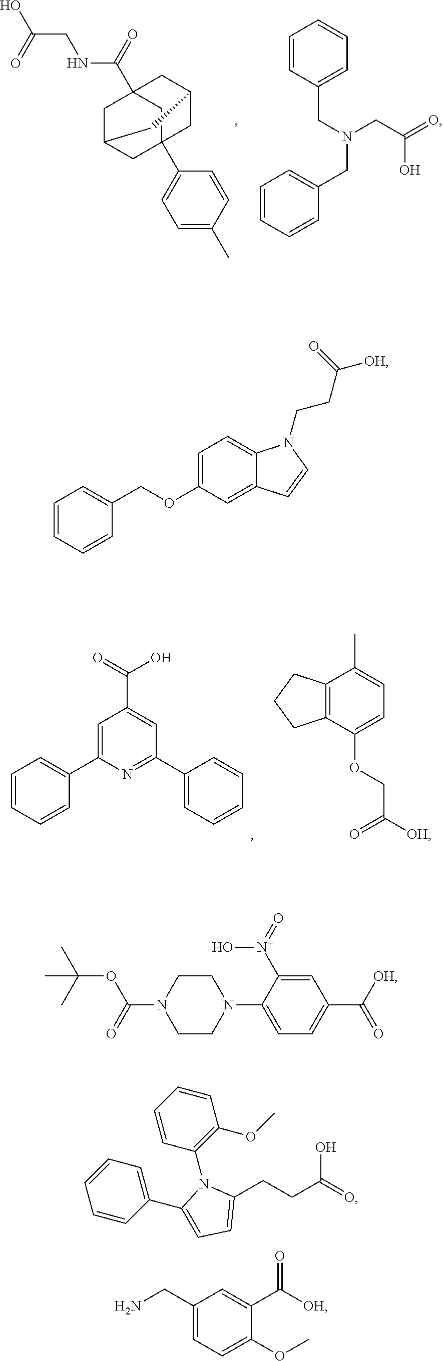

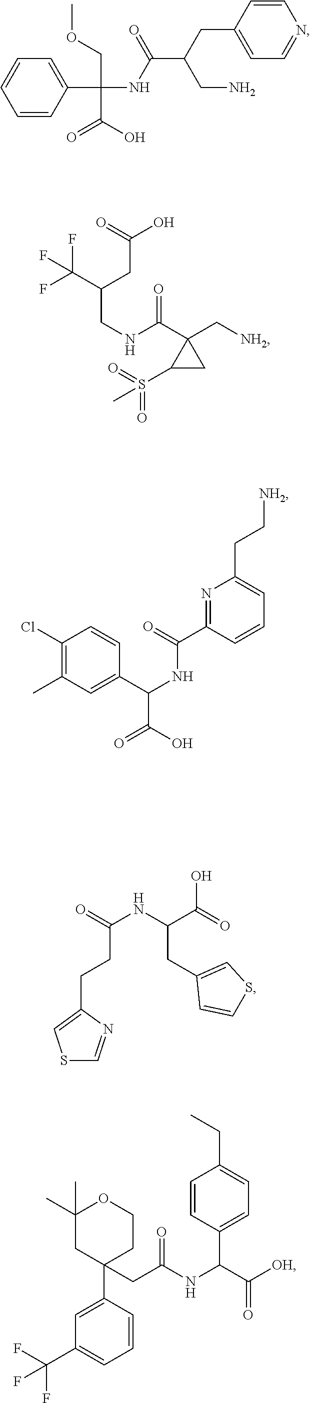 [SMILES: COCC(NC(=O)C(CN)Cc1ccncc1)(C(=O)O)c1ccccc1 ; CS(=O)(=O)C1CC1(CN)C(=O)NCC(CC(=O)O)C(F)(F)F ; Cc1cc(C(NC(=O)c2cccc(CCN)n2)C(=O)O)ccc1Cl ; O=C(CCc1cscn1)NC(Cc1ccsc1)C(=O)O ; CCc1ccc(C(NC(=O)CC2(c3cccc(C(F)(F)F)c3)CCOC(C)(C)C2)C(=O)O)cc1]

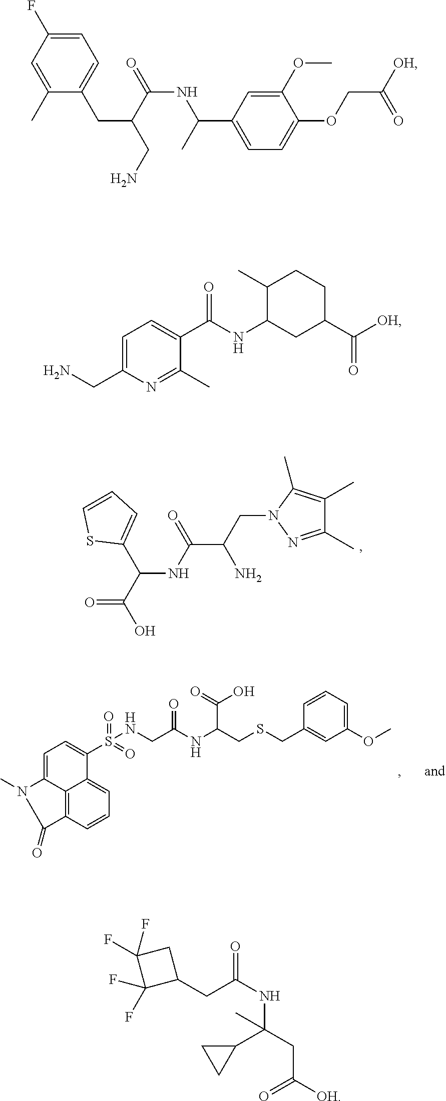

Preferably, the small molecule is selected from:

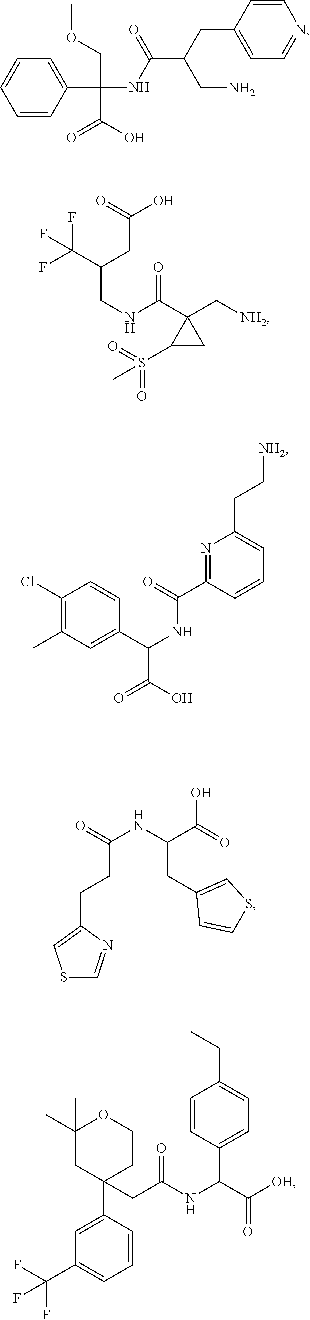 [SMILES: COCC(NC(=O)C(CN)Cc1ccncc1)(C(=O)O)c1ccccc1 ; CS(=O)(=O)C1CC1(CN)C(=O)NCC(CC(=O)O)C(F)(F)F ; Cc1cc(C(NC(=O)c2cccc(CCN)n2)C(=O)O)ccc1Cl ; O=C(CCc1cscn1)NC(Cc1ccsc1)C(=O)O ; CCc1ccc(C(NC(=O)CC2(c3cccc(C(F)(F)F)c3)CCOC(C)(C)C2)C(=O)O)cc1]

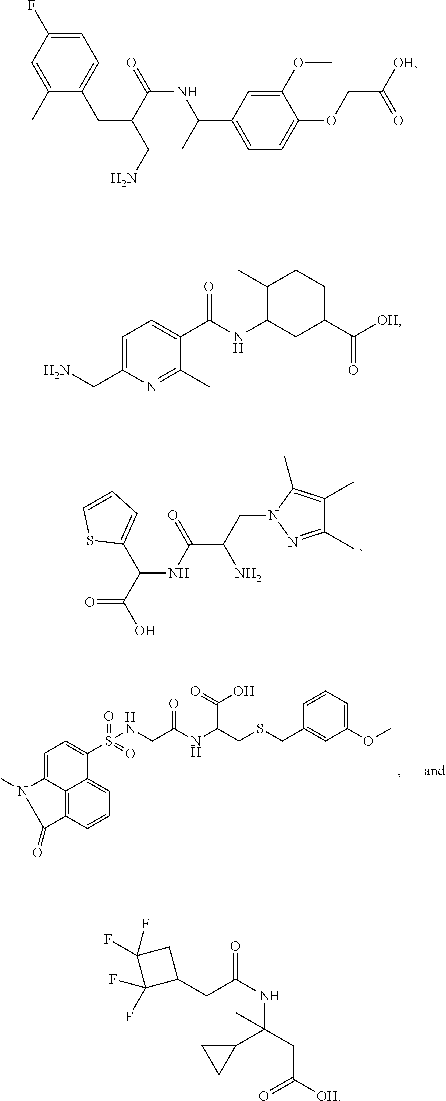

In some embodiments, the small molecule is selected from lumacaftor (VX-809), ivacaftor (VX-770), tezacaftor and elexacaftor.

In some embodiments, the linker is an alkyl, a polyethylene glycol (PEG) or other similar molecule, or a click linker. As used herein, the “alkyl” may be branched or linear, substituted or unsubstituted. The length of the alkyl is selected to maximize, or at least not substantially interfere with the efficient binding of the DUB binder and the target binder. For example, the “alkyl” may be C₁-C₂₅, such as C₁-C₂₀, including C₁-C₁₅, and C₁-C₅. Thus, the alkyl linker may include C1, C2, C3, C4, C5, C6, C7, C8, C9, C10, C11, C12, C13, C14, C15, C16, C17, C18, C19, C20, C21, C22, C23, C24, C25 or higher carbon chain. As used herein a “click linker” is a class of biocompatible small molecules that are used in bioconjugation, allowing the joining of substrates of choice with specific biomolecules. It is based on “click” chemistry which is fully desctribed in Kolb et al. (2001) “Click Chemistry: Diverse Chemical Function from a Few Good Reactions”. Angewandte Chemie International Edition. 40 (11): 2004-2021.

Another embodiment of the present disclosure is a method of treating or ameliorating the effects of a disease in a subject, comprising administering to the subject an effective amount of a bivalent molecule disclosed herein.

In some embodiments, the subject is a human. In some embodiments, the disease is selected from the group consisting of an inherited ion channelopathy, a cancer, a cardiovascular condition, an infectious disease, and a metabolic disease. In some embodiments, the inherited ion channelopathy is selected from the group consisting of epilepsy, migraine, neuropathic pain, cardiac arrhythmias, long QT syndrome, Brugada syndrome, cystic fibrosis, diabetes, hyperinsulinemic hypoglycemia, Bartter syndrome, and diabetes insipidus. In some embodiments, the inherited ion channelopathy is cystic fibrosis.

As used herein, the terms “treat,” “treating,” “treatment” and grammatical variations thereof mean subjecting an individual subject to a protocol, regimen, process or remedy, in which it is desired to obtain a physiologic response or outcome in that subject, e.g., a patient. However, because every treated subject may not respond to a particular treatment protocol, regimen, process or remedy, treating does not require that the desired physiologic response or outcome be achieved in each and every subject or subject population, e.g., patient population. Accordingly, a given subject or subject population, e.g., patient population may fail to respond or respond inadequately to treatment.

As used herein, the terms “ameliorate”, “ameliorating” and grammatical variations thereof mean to decrease the severity of the symptoms of a disease in a subject, preferably a human.

As used herein, “administration,” “administering” and variants thereof means introducing a composition, such as a synthetic membrane-receiver complex, or agent into a subject and includes concurrent and sequential introduction of a composition or agent. The introduction of a composition or agent into a subject is by any suitable route, including orally, pulmonarily, intranasally, parenterally (intravenously, intramuscularly, intraperitoneally, or subcutaneously), rectally, intralymphatically, or topically. Administration includes self-administration and the administration by another. A suitable route of administration allows the composition or the agent to perform its intended function. For example, if a suitable route is intravenous, the composition is administered by introducing the composition or agent into a vein of the subject. Administration can be carried out by any suitable route.

As used herein, a “subject” is a mammal, preferably, a human. In addition to humans, categories of mammals within the scope of the present disclosure include, for example, farm animals, domestic animals, laboratory animals, etc. Some examples of farm animals include cows, pigs, horses, goats, etc. Some examples of domestic animals include dogs, cats, etc. Some examples of laboratory animals include primates, rats, mice, rabbits, guinea pigs, etc.

Still another embodiment of the present disclosure is a method of identifying and preparing a small molecule binder targeting a protein of interest, comprising: a) generating a DNA-encoded compound library; b) incubating the library with the protein of interest; c) washing off unbound molecules; d) amplifying the oligonucleotide codes of the binding compounds by PCR and constructing an enriched compound library; e) repeating steps b) to d) with the enriched library as necessary to further enrich the library containing the oligonucleotide codes of the binding compounds; and f) identifying the small molecule binders by decoding the library generated in step e) for binding validation.

In some embodiments, the protein of interest is cystic fibrosis transmembrane conductance regulator (CFTR). In some embodiments, the protein of interest is a deubiquitinase (DUB).

In some embodiments, the DNA-encoded compound library is generated by a technique that is non-evolution-based or evolution-based. Non-limiting examples of non-evolution-based techniques include “split-and-pool” method and Encoded Self-Assembling Chemical (ESAC) technology. Non-limiting examples of evolution-based techniques include DNA-routing, DNA-templated synthesis, and YoctoReactor technology. In some embodiments, the decoding in step f) is carried out by Sanger sequencing, microarray, or high throughput sequencing.

### Additional Definitions

As used herein, the terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymers.

“Nucleic acid” or “oligonucleotide” or “polynucleotide” used herein means at least two nucleotides covalently linked together. Many variants of a nucleic acid may be used for the same purpose as a given nucleic acid. Thus, a nucleic acid also encompasses substantially identical nucleic acids and complements thereof.

Nucleic acids may be single stranded or double stranded, or may contain portions of both double stranded and single stranded sequences. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be synthesized as a single stranded molecule or expressed in a cell (in vitro or in vivo) using a synthetic gene. Nucleic acids may be obtained by chemical synthesis methods or by recombinant methods.

The nucleic acid may also be an RNA such as an mRNA, tRNA, short hairpin RNA (shRNA), short interfering RNA (siRNA), double-stranded RNA (dsRNA), transcriptional gene silencing RNA (ptgsRNA), Piwi-interacting RNA, pri-miRNA, pre-miRNA, micro-RNA (miRNA), or anti-miRNA.

“Vector” used herein refers to an assembly which is capable of directing the expression of desired protein. The vector must include transcriptional promoter elements which are operably linked to the gene(s) of interest. The vector may be composed of either deoxyribonucleic acids (“DNA”), ribonucleic acids (“RNA”), or a combination of the two (e.g., a DNA-RNA chimeric). Optionally, the vector may include a polyadenylation sequence, one or more restriction sites, as well as one or more selectable markers such as neomycin phosphotransferase or hygromycin phosphotransferase. Additionally, depending on the host cell chosen and the vector employed, other genetic elements such as an origin of replication, additional nucleic acid restriction sites, enhancers, sequences conferring inducibility of transcription, and selectable markers, may also be incorporated into the vectors described herein.

As used herein, the terms “cell”, “host cell” or “recombinant host cell” refers to host cells that have been engineered to express a desired recombinant protein. Methods of creating recombinant host cells are well known in the art. For example, see Sambrook et al. (MOLECULAR CLONING: A LABORATORY MANUAL (Sambrook et al, eds., Cold Spring Harbor Laboratory Press, Cold Spring Harbor, 1989), Ausubel et al. (CURRENT PROTOCOLS IN MOLECULAR BIOLOGY Ausubel et al., eds., John Wiley & Sons, New York, 1987). In the present disclosure, the host cells are transformed with the vectors described herein.

Recombinant host cells as used herein may be any of the host cells used for recombinant protein production, including, but not limited to, bacteria, yeast, insect and mammalian cell lines.

As used herein, the term “increase,” “enhance,” “stimulate,” and/or “induce” (and like terms) generally refers to the act of improving or increasing, either directly or indirectly, a concentration, level, function, activity, or behavior relative to the natural, expected, or average, or relative to a control condition.

As used herein, the term “inhibit,” “suppress,” “decrease,” “interfere,” and/or “reduce” (and like terms) generally refers to the act of reducing, either directly or indirectly, a concentration, level, function, activity, or behavior relative to the natural, expected, or average, or relative to a control condition.

The terminology used herein is for the purpose of describing particular embodiments only and is not intended to be limiting. As used in the specification and the appended claims, the singular forms “a,” “an,” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

The following examples are provided to further illustrate certain aspects of the present disclosure. These examples are illustrative only and are not intended to limit the scope of the disclosure in any way.

### EXAMPLES

### Example 1

### RESTORx: A NEXT-GENERATION THERAPEUTIC MODALITY BASED ON TARGETED PROTEIN STABILIZATION

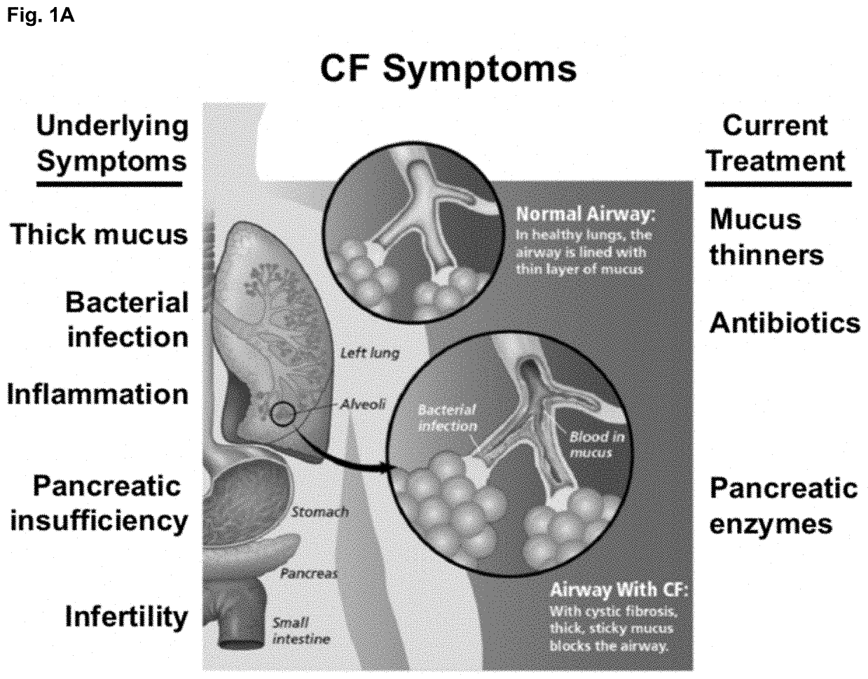

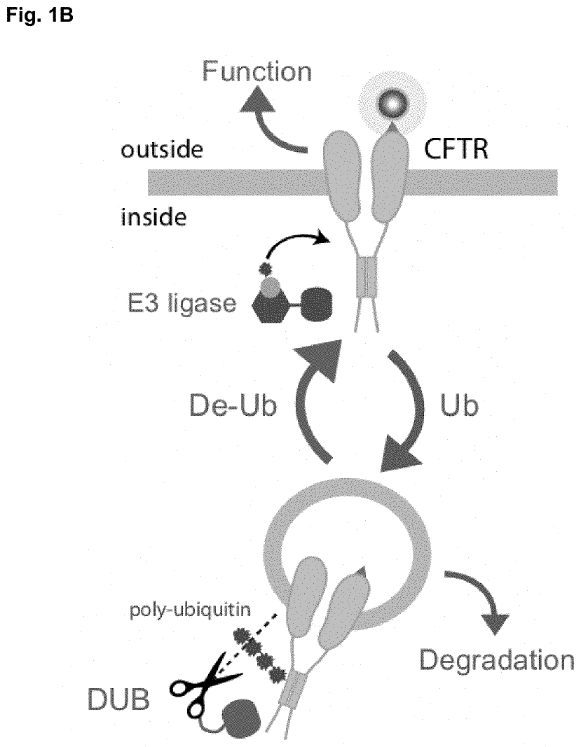

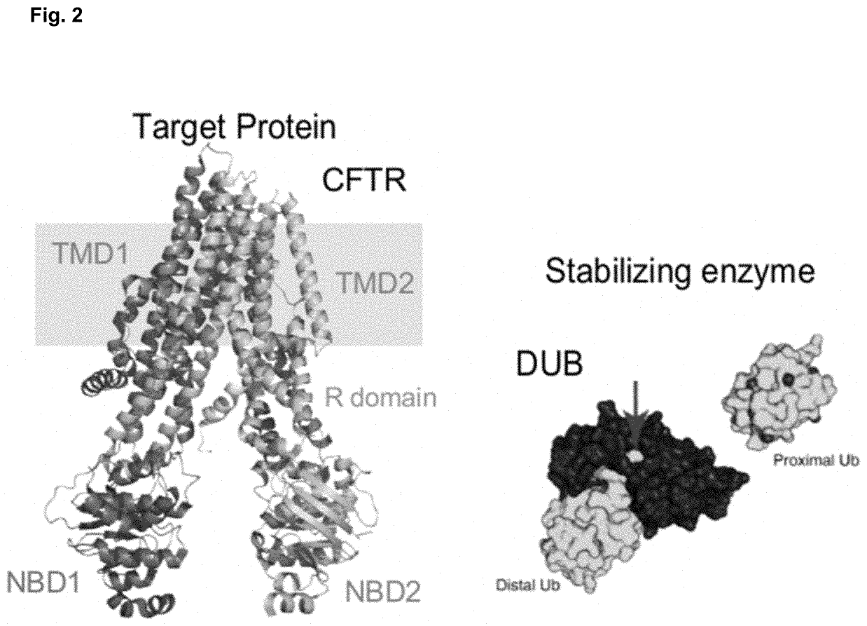

Protein stability is a key point of regulation for all proteins in the cell. Ubiquitination plays a major role in intracellular protein homeostasis, and dysregulation of this process can lead to the pathogenesis of many diseases. The present disclosure focuses on cystic fibrosis (CF), a rare, inherited disease with high unmet need, as the primary indication. Although the vast majority of CF mutations lead to deficits in the stability of a chloride channel, CFTR, the current gold standard treatments are overwhelmingly symptom based: lung airway clearance techniques, inhalation of mucus thinners, and antibiotic treatment of bacterial infections (FIGS. 1A-1B). While these treatments have improved life expectancy (˜-30-40 years old), there remains no definitive treatment and CF patients continue to experience rapidly deteriorating quality of life. Only recently has there been a push in the development of pharmacologic chaperones, or “correctors”, that look to promote mutant CFTR trafficking to the cell membrane; however, to date, the clinical efficacy of such treatments has been relatively modest with many mutations remaining resistant to therapy.

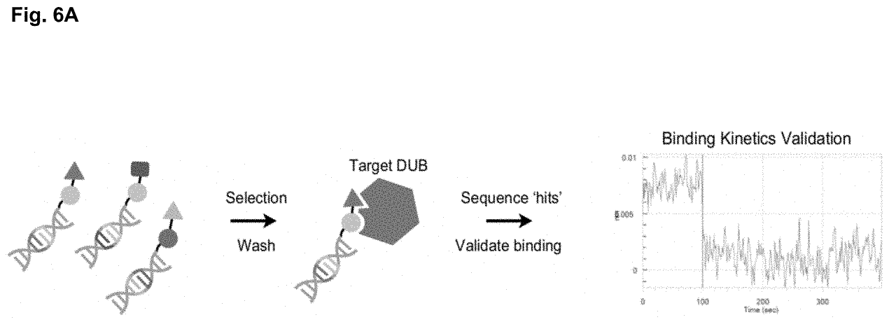

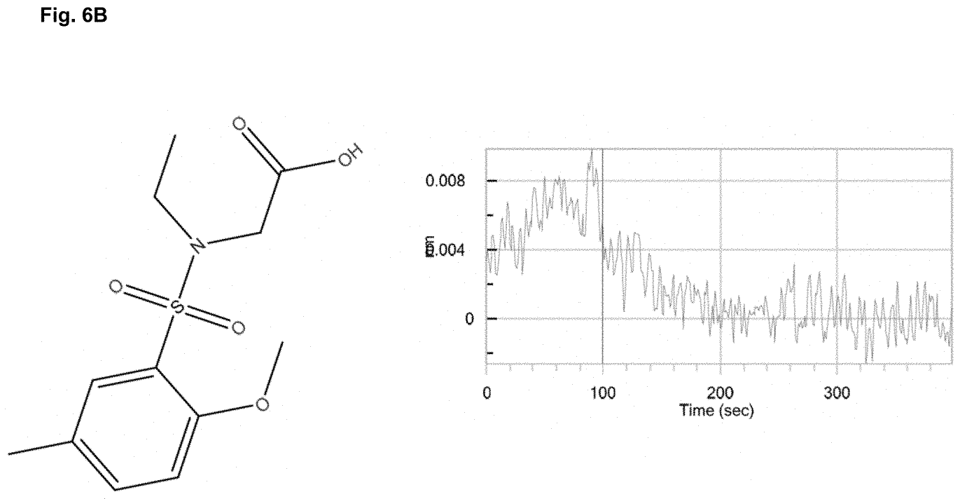

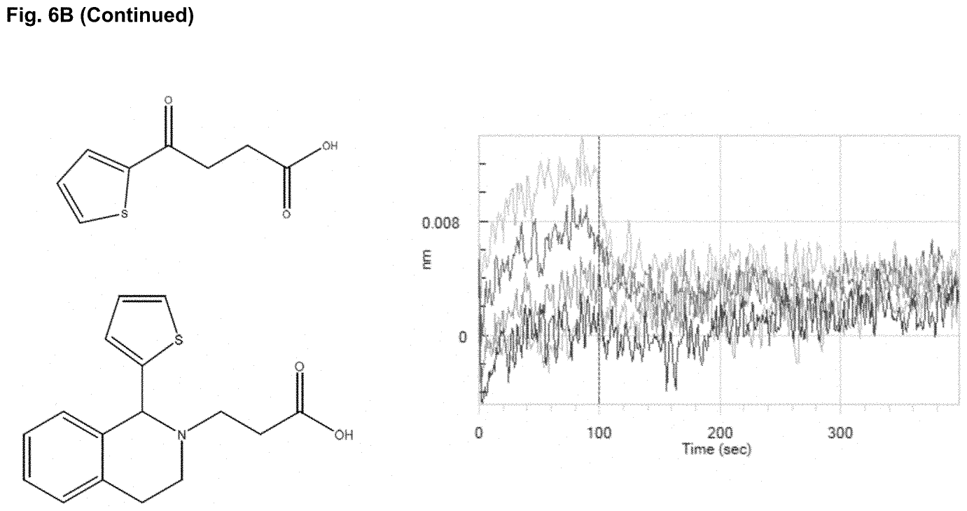

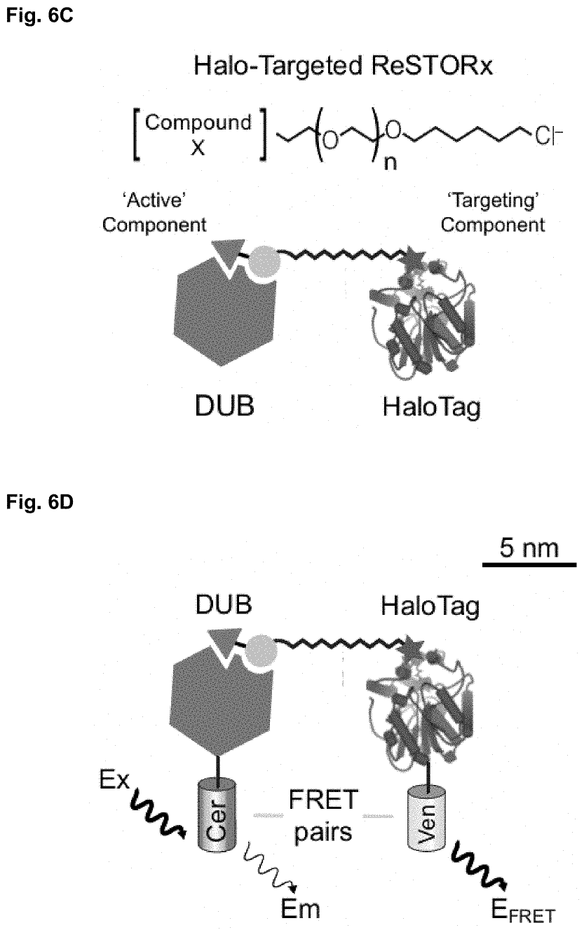

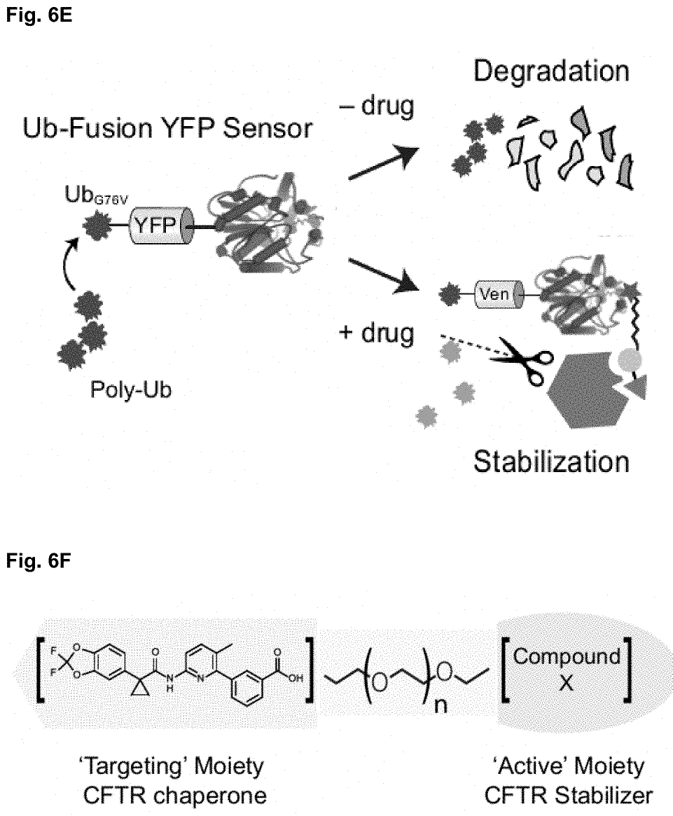

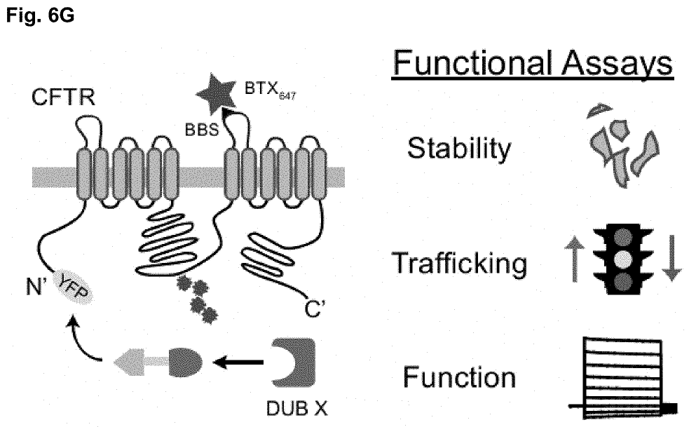

The present disclosure took an entirely distinct small-molecule approach for the rescue of CFTR trafficking and stability (FIG. 2). In particular, the goal was to exploit the powerful, yet reversible nature of ubiquitination with a novel hypothesis: could we recruit endogenous deubiquitinases (DUBs) to mutant CFTR channels in order to selectively tune the ubiquitin status, enhance channel stability, and restore function? We term this general approach Rescue & Stabilization on Redirection of Endogenous DUBs (ReSTORED), and resulting molecules that exploit this mechanism, called Rescue and Stabilization Therapeutics (ReSTORx). Fundamentally, our ReSTORx are heterobifunctional molecules comprised of 3 distinct modules: 1) a DUBbinding molecule, 2) a target-binding molecule, and 3) a variable linker joining the two. As such, our ReSTORx compounds act as molecular bridges, joining endogenous DUB activity to a target protein-of-interest. To test this novel approach, we generated an “all small-molecule” ReSTORx tool compound (FIG. 3 and FIGS. 6A-6G). Initial screens have uncovered a variety of putative hits representing new chemical matter for DUB binders. The research roadmap was as follows: 1) Hit-to-lead development of an “active” DUB-recruiting ReSTORx component using target engagement and stabilization assays in living cells, and 2) Modification of the CFTR modulator, lumacaftor, as a “targeting” component for the first “all small-molecule” CF ReSTORx compound.

The ReSTORx technology emerges as a first-in-class CFTR stabilizer, distinct from any therapeutics on market or in development for CF, and rationally designed for targeted ubiquitin removal from mutant channels. Its unique mechanism-of-action promotes synergistic efficacy with current modulators, and rescues previously unresponsive CFTR mutations. Furthermore, the modular nature of the ReSTORx technology suggests a highly adaptable, protein stabilizing platform. As such, the “active” DUB-recruiting components can be readily adapted for use with any given target-binding molecule, with the potential for improving the efficacy of currently marketed drugs or functionalizing previously quiescent compounds that engage a target without therapeutic effect.

The potential impact of such a ReSTORx platform extends into the ubiquitin therapeutic space. Competition in ubiquitin therapeutics has been mainly confined to nonselective inhibitors of the ubiquitin proteasome system (UPS). Proteasome inhibitors have had large commercial success, for example, the first-to-market UPS modulator, Velcade® (bortezomib), generated $3 billion USD revenue in 2014 alone; however, since these drugs target the entire protein degradation pathway, lack of target specificity has restricted their use and led to significant side effects in patients. Consequently, the focus is gradually shifting from proteasome inhibitors to targeting specific components of the UPS (i.e. E3 ubiquitin ligases). However, even these ubiquitin enzymes suffer from promiscuity in the regulation of many different substrates. In contrast, the ReSTORx molecules disclosed herein enjoy both specificity in targeting and generalizability in action, exploiting a huge unmet market need for selective UPS modulators. This entirely new therapeutic modality can further expand indications to other inherited channelopathies and cancer therapeutics.

### Hit-to-Lead Development of an “Active” DUB-Recruiting ReSTORx Component Using Target Engagement and Stabilization Assays in Living Cells

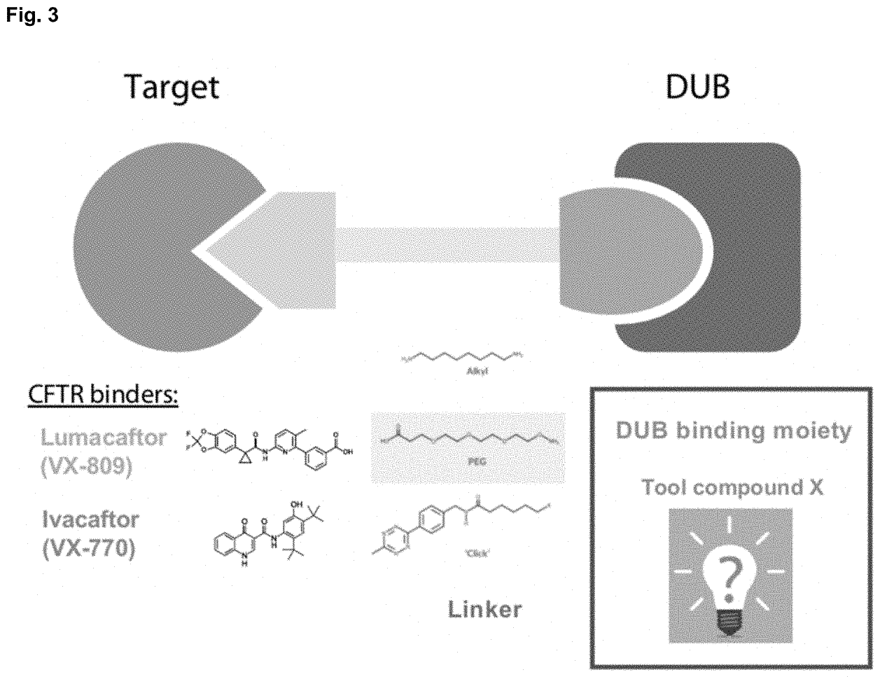

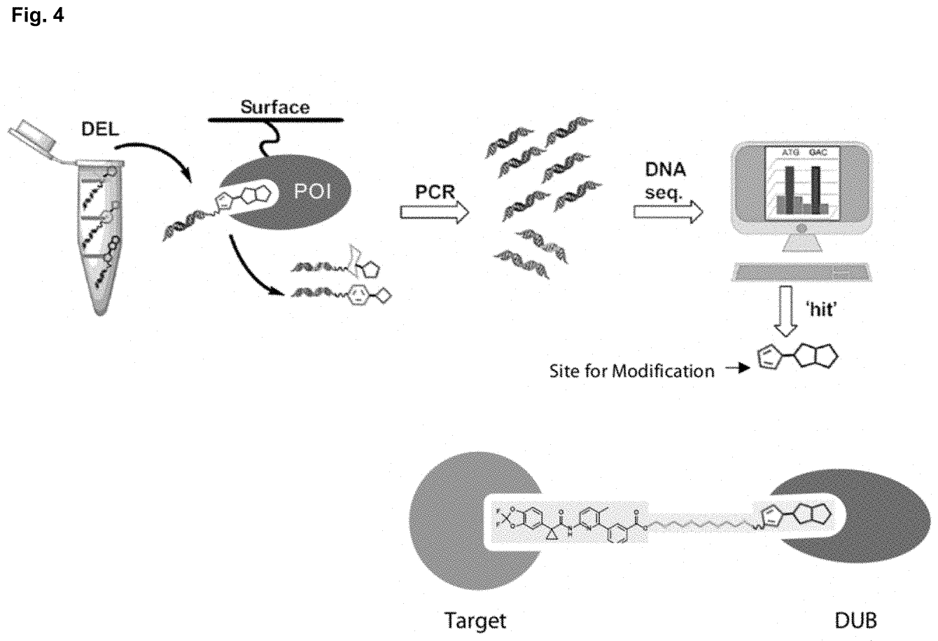

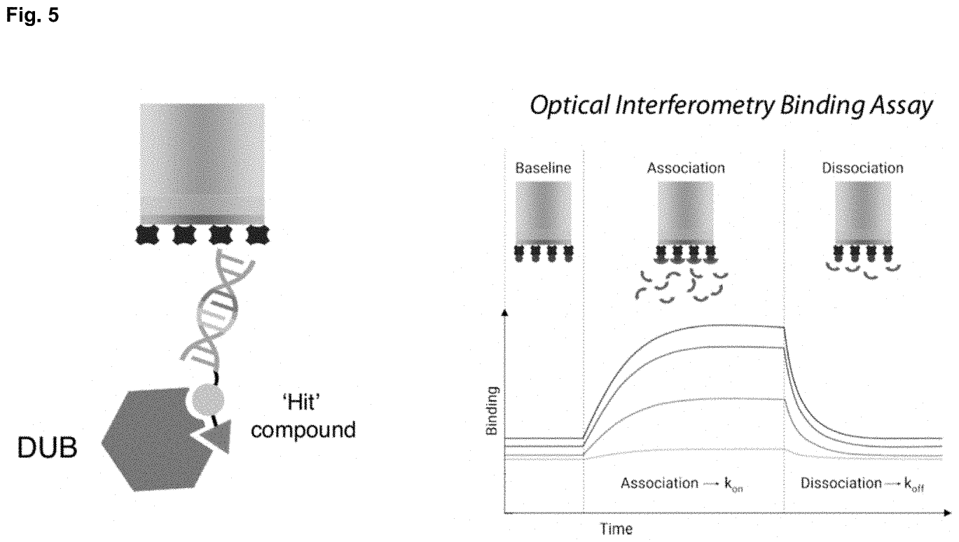

We have acquired putative, small-molecule DUB binders using proprietary DNA-encoded compound library technology (FIG. 4). The initial hits were validated using cell-free binding assays to determine Kds, as well as Kon/Koff rates (FIG. 5). Next, we received top hits for screening and development in target engagement assays in the cellular environment. To begin, validated hits were resynthesized with HaloTag ligand (chloroalkane) at the site of previous DNA-attachment in consultation with the OCCC. The benefit of using a HaloTag ligand is the modular ability to target our “active” moiety to any HaloTag-fused protein. As a result, the choloroalkane moiety effectively acted as our “targeting” component for our ReSTORx molecules. Next, we utilized a series of validation assays in living cells to develop our lead “active” component for DUB-recruitment and target stabilization. First, we tested target engagement of each hit compound using a FRET assay (FIG. 6D). Second, we tested the functional deubiquitination efficacy of our hit compounds using a ubiquitin-dependent, destabilized YFPHaloTag assay (FIG. 6E). Under basal conditions, this YFP fluorescence will be minimal as the reporter is constantly poly-ubiquitinated and degraded. However, upon recruitment of a DUB with an “active” ReSTORx component, any poly-ubiquitin chains will be removed and YFP fluorescence will be stabilized.

### Modification of the CFTR Modulator, Lumacaftor, as a “Targeting Component for the First “All Smallmolecule” CF ReSTORx Compound

We have addressed the “active” component and backbone of our ReSTORx platform: the DUBrecruiting moiety necessary for ubiquitin removal. A competitive advantage of this platform is the ability to functionalize any target binder with this “active” component. The development of CFTR modulators for cystic fibrosis provides a perfect starting point for validation studies. Notably, several groups have demonstrated the direct action of lumacaftor through binding mutant CFTR channels. In particular, a previous study modified lumacaftor itself, inserting a functional alkyne group that could be labeled with biotin-azide using click chemistry, and biochemically isolated using streptavidin beads. Importantly, this alkyne modification did not affect the rescuing capacity of lumacaftor and allowed for the robust purification of lumacaftor-bound CFTR channels from living cells. Lumacaftor was used as a bona fide CFTR binder by incorporating it as a “targeting” component in our first “all small-molecule” CF ReSTORx compound. We utilized the OCCC for the modification of lumacaftor and synthesis of our lead DUB-recruiting “active” component in tandem. During synthesis, we generated several linker lengths (with varying ethylene glycol repeats) to determine optimal performance. We then tested the efficacy of these complete CF-targeted ReSTORx molecules using a series of complementary cellular CFTR assays in house to determine rescue of stability, trafficking, and function.

### DOCUMENTS CITED

The following references, to the extent that they provide exemplary procedural or other details supplementary to those set forth herein, are specifically incorporated herein by reference.

1. Kullmann, D. M. Neurological Channelopathies. Annual Review of Neuroscience 33, 151-172, doi:10.1146/annurev-neuro-060909-153122 (2010).

2. Bohnen, M. S. et al. Molecular Pathophysiology of Congenital Long QT Syndrome. Physiological Reviews 97, 89-134, doi:10.1152/physrev.00008.2016 (2016).

3. Cutting, G. R. Cystic fibrosis genetics: from molecular understanding to clinical application. Nature Reviews Genetics 16, 45-56, doi:10.1038/nrg3849 (2014).

4. Curran, J. & Mohler, P. J. Alternative Paradigms for Ion Channelopathies: Disorders of Ion Channel Membrane Trafficking and Posttranslational Modification. Annual Review of Physiology 77, 1-20, doi: 10.1146/annurev-physiol-021014-071838 (2015).

5. Foot, N., Henshall, T. & Kumar, S. Ubiquitination and the Regulation of Membrane Proteins. Physiol Rev 97, 253-281, doi:10.1152/physrev.00012.2016 (2017).

6. MacGurn, J. A., Hsu, P. C. & Emr, S. D. Ubiquitin and membrane protein turnover: from cradle to grave. Annu Rev Biochem 81, 231-259, doi:10.1146/annurevbiochem-060210-093619 (2012).

7. Ashcroft, F. M. & Rorsman, P. KATP channels and islet hormone secretion: new insights and controversies. Nature Reviews Endocrinology 9, 660, doi:10.1038/nrendo.2013.166 (2013).

8. Imbrici, P. et al. Therapeutic Approaches to Genetic Ion Channelopathies and Perspectives in Drug Discovery. Frontiers in Pharmacology 7, 121, doi:10.3389/fphar.2016.00121 (2016).

9. Wulff, H., Christophersen, P., Colussi, P., Chandy, G. K. & Yarov-Yarovoy, V. Antibodies and venom peptides: new modalities for ion channels. Nature Reviews Drug Discovery, 1, doi:10.1038/s41573-019-0013-8 (2019).

10. Tester, D. J., Will, M. L., Haglund, C. M. & Ackerman, M. J. Compendium of cardiac channel mutations in 541 consecutive unrelated patients referred for long QT syndrome genetic testing. Heart rhythm 2, 507-517, doi:10.1016/j.hrthm.2005.01.020 (2005).

11. Wilson, A. J., Quinn, K. V., Graves, F. M., Bitner-Glindzicz, M. & Tinker, A. Abnormal KCNQ1 trafficking influences disease pathogenesis in hereditary long QT syndromes (LQT1). Cardiovascular Research 67, 476-486, doi:10.1016/j.cardiores.2005.04.036 (2005).

12. Haardt, M., Benharouga, M., Lechardeur, D., Kartner, N. & Lukacs, G. L. C-terminal truncations destabilize the cystic fibrosis transmembrane conductance regulator without impairing its biogenesis. A novel class of mutation. The Journal of biological chemistry 274, 21873-21877, doi:10.1074/jbc.274.31.21873 (1999).

13. Cheng, S. H. et al. Defective intracellular transport and processing of CFTR is the molecular basis of most cystic fibrosis. Cell 63, 827-834 (1990).

14. Nalepa, G., Rolfe, M. & Harper, W. J. Drug discovery in the ubiquitin-proteasome system. Nature Reviews Drug Discovery 5, 596-613, doi:10.1038/nrd2056 (2006).

15. Huang, X. & Dixit, V. M. Drugging the undruggables: exploring the ubiquitin system for drug development. Cell Research 26, 484-498, doi:10.1038/cr.2016.31 (2016).

16. Jespersen, T. et al. The KCNQ1 potassium channel is down-regulated by ubiquitylating enzymes of the Nedd4/Nedd4-like family. Cardiovasc Res 74, 64-74, doi:10.1016/j.cardiores.2007.01.008 (2007).

17. Mevissen, T. et al. OTU Deubiquitinases Reveal Mechanisms of Linkage Specificity and Enable Ubiquitin Chain Restriction Analysis. Cell 154, 169-184, doi:10.1016/j.ce11.2013.05.046 (2013).

18. Rothbauer, U. et al. A versatile nanotrap for biochemical and functional studies with fluorescent fusion proteins. Molecular & cellular proteomics : MCP 7, 282-289, doi:10.1074/mcp.M700342-MCP200 (2008).

19. Aromolaran, A. S., Subramanyam, P., Chang, D. D., Kobertz, W. R. & Colecraft, H. M. LQT1 mutations in KCNQ1 C-terminus assembly domain suppress IKs using different mechanisms. Cardiovasc Res 104, 501-511, doi:10.1093/cvr/cvu231 (2014).

20. Peroz, D., Dahimène, S., Baró, I., Loussouarn, G. & Mérot, J. LQT1-associated Mutations Increase KCNQ1 Proteasomal Degradation Independently of Derlin-1. Journal of Biological Chemistry 284, 5250-5256, doi:10.1074/jbc.M806459200 (2009).

21. Mattmann, M. E. et al. Identification of (R)-N-(4-(4-methoxyphenyl)thiazol-2-yl)-1-tosylpiperidine-2-carboxamide, ML277, as a novel, potent and selective Kv7.1 (KCNQ1) potassium channel activator. Bioorganic & Medicinal Chemistry Letters 22, 5936-5941, doi:10.1016/j.bmc1.2012.07.060 (2012).

22. Veit, G. et al. From CFTR biology toward combinatorial pharmacotherapy: expanded classification of cystic fibrosis mutations. Molecular biology of the cell 27, 424-433, doi:10.1091/mbc.E14-04-0935 (2016).

23. Boeck, K. & Amaral, M. D. Progress in therapies for cystic fibrosis. The Lancet Respiratory Medicine 4, 662-674, doi: 10.1016/S2213-2600(16)00023-0 (2016).

24. Wainwright, C. E. et al. Lumacaftor-Ivacaftor in Patients with Cystic Fibrosis Homozygous for Phe508del CFTR. The New England Journal of Medicine 373, 220-231, doi:10.1056/NEJMoa1409547 (2015).

25. Goor, F. et al. Correction of the F508del-CFTR protein processing defect in vitro by the investigational drug VX-809. Proceedings of the National Academy of Sciences 108, 18843-18848, doi:10.1073/pnas.1105787108 (2011).

26. Goor, F. et al. Rescue of CF airway epithelial cell function in vitro by a CFTR potentiator, VX-770. Proceedings of the National Academy of Sciences 106, 18825-18830, doi:10.1073/pnas.0904709106 (2009).

27. Farinha, C. M. & Matos, P. Repairing the basic defect in cystic fibrosis—one approach is not enough. FEBS Journal 283, 246-264, doi:10.1111/febs.13531 (2016).

28. Faesen, A. C. et al. The differential modulation of USP activity by internal regulatory domains, interactors and eight ubiquitin chain types. Chem Biol 18, 1550-1561, doi:10.1016/j.chembio1.2011.10.017 (2011).

29. McMahon, C. et al. Yeast surface display platform for rapid discovery of conformationally selective nanobodies. Nature Structural & Molecular Biology 25, 289-296, doi:10.1038/s41594-018-0028-6 (2018).

30. Galietta, L. V., Jayaraman, S. & Verkman, A. S. Cell-based assay for highthroughput quantitative screening of CFTR chloride transport agonists. American journal of physiology. Cell physiology 281, 42, doi:10.1152/ajpce11.2001.281.5.C1734 (2001).

31. Yu, H. et al. Ivacaftor potentiation of multiple CFTR channels with gating mutations. Journal of Cystic Fibrosis 11, 237-245, doi:10.1016/j.jcf.2011.12.005 (2012).

32. Ratner, M. FDA deems in vitro data on mutations sufficient to expand cystic fibrosis drug label. Nature Biotechnology 35, 606-606, doi:10.1038/nbt0717-606 (2017).

33. Durmowicz, A. G., Lim, R., Rogers, H., Rosebraugh, C. J. & Chowdhury, B. A. The

U.S. Food and Drug Administration's Experience with Ivacaftor in Cystic Fibrosis. Establishing Efficacy Using In Vitro Data in Lieu of a Clinical Trial. Annals of the American Thoracic Society 15, 1-2, doi:10.1513/AnnalsATS.201708-668PS (2018).

34. Han, S. T. et al. Residual function of cystic fibrosis mutants predicts response to small molecule CFTR modulators. JCI Insight 3, doi:10.1172/jci.insight.121159 (2018).

35. Goor, F., Yu, H., Burton, B. & Hoffman, B. J. Effect of ivacaftor on CFTR forms with missense mutations associated with defects in protein processing or function. Journal of Cystic Fibrosis 13, 29-36, doi:10.1016/j.jcf.2013.06.008 (2014).

36. Lukacs, G. L. & Verkman, A. S. CFTR: folding, misfolding and correcting the ΔF508 conformational defect. Trends in Molecular Medicine 18, 81-91, doi:10.1016/j.molmed.2011.10.003 (2012).

37. Okiyoneda, T. et al. Mechanism-based corrector combination restores ΔF508-CFTR folding and function. Nature Chemical Biology 9, 444-454, doi:10.1038/nchembio.1253 (2013).

38. Okiyoneda, T. et al. Peripheral protein quality control removes unfolded CFTR from the plasma membrane. Science (New York, N.Y.) 329, 805-810, doi:10.1126/science.1191542 (2010).

39. Sigoillot, M. et al. Domain-interface dynamics of CFTR revealed by stabilizing nanobodies. Nature Communications 10, doi:ARTN 263610.1038/s41467-019-10714-y (2019).

40. Delisle, B. P. et al. Biology of Cardiac Arrhythmias. Circulation Research 94, 1418-1428, doi:10.1161/01.RES.0000128561.28701.ea (2004).

41. Liu, F., Zhang, Z., Csanady, L., Gadsby, D. C. & Chen, J. Molecular Structure of the Human CFTR Ion Channel. Cell 169, 85-910065408, doi:10.1016/j.ce11.2017.02.024 (2017).

42. Kanner, S. A., Morgenstern, T. & Colecraft, H. M. Sculpting ion channel functional expression with engineered ubiquitin ligases. eLife 6, doi:10.7554/eLife.29744 (2017).

43. Fridy, P. C. et al. A robust pipeline for rapid production of versatile nanobody repertoires. Nature Methods 11, 1253-1260, doi: 10.1038/nm eth.3170 (2014).

44. Sekine-Aizawa, Y. & Huganir, R. L. Imaging of receptor trafficking by using alphabungarotoxin-binding-site-tagged receptors. Proc Natl Acad Sci U S A 101, 17114-17119, doi:10.1073/pnas.0407563101 (2004).

45. Gao, S. et al. Ubiquitin ligase Nedd4L targets activated Smad2/3 to limit TGF-beta signaling. Mol Cell 36, 457-468, doi:10.1016/j.molce1.2009.09.043 (2009).

46. Peters, K. W. et al. CFTR Folding Consortium: methods available for studies of CFTR folding and correction. Methods Mol Biol 742, 335-353, doi:10.1007/978-1-61779-120-8_20 (2011).

47. Galietta, L. J., Haggie, P. M. & Verkman, A. S. Green fluorescent protein-based halide indicators with improved chloride and iodide affinities. FEBS letters 499, 220-224, doi:10.1016/s0014-5793(01)02561-3 (2001).

48. Kanner, S. A., Jain, A. & Colecraft, H. M. Development of a High-Throughput Flow Cytometry Assay to Monitor Defective Trafficking and Rescue of Long QT2 Mutant hERG Channels. Frontiers in Physiology 9, 397, doi:10.3389/fphys.2018.00397 (2018).

49. Lee, S.-R., Sang, L. & Yue, D. T. Uncovering Aberrant Mutant PKA Function with Flow Cytometric FRET. Cell Reports 14, 3019-3029, doi:10.1016/j.celrep.2016.02.077 (2016).

All patents, patent applications, and publications cited herein are incorporated herein by reference in their entirety as if recited in full herein.

The disclosure being thus described, it will be obvious that the same may be varied in many ways. Such variations are not to be regarded as a departure from the spirit and scope of the disclosure and all such modifications are intended to be included within the scope of the following claims.

What is claimed is:

1. A bivalent molecule comprising:
a) a deubiquitinase (DUB) binder;
b) a target binder; and
c) a variable linker between the DUB binder and the target binder, wherein the DUB binder is a small molecule.

2. The bivalent molecule of claim 1, wherein the DUB is endogenous.

3. The bivalent molecule of claim 1, wherein the DUB is selected from the ubiquitin specific proteases (USP) family, the ovarian tumor proteases (OTU) family, the ubiquitin C-terminal hydrolases (UCH) family, the Josephin domain family (Josephin), the motif interacting with ubiquitin-containing novel DUB family (MINDY), and the JAB1/MPN/Mov34 metalloenzyme domain family (JAMM).

4. The bivalent molecule of claim 1, wherein the DUB is USP21 or USP2.

5. The bivalent molecule of claim 1, wherein the small molecule binds to a USP family member.

6. The bivalent molecule of claim 1, wherein the small molecule binds to a USP2.

7. The bivalent molecule of claim 1, wherein the small molecule is selected from:

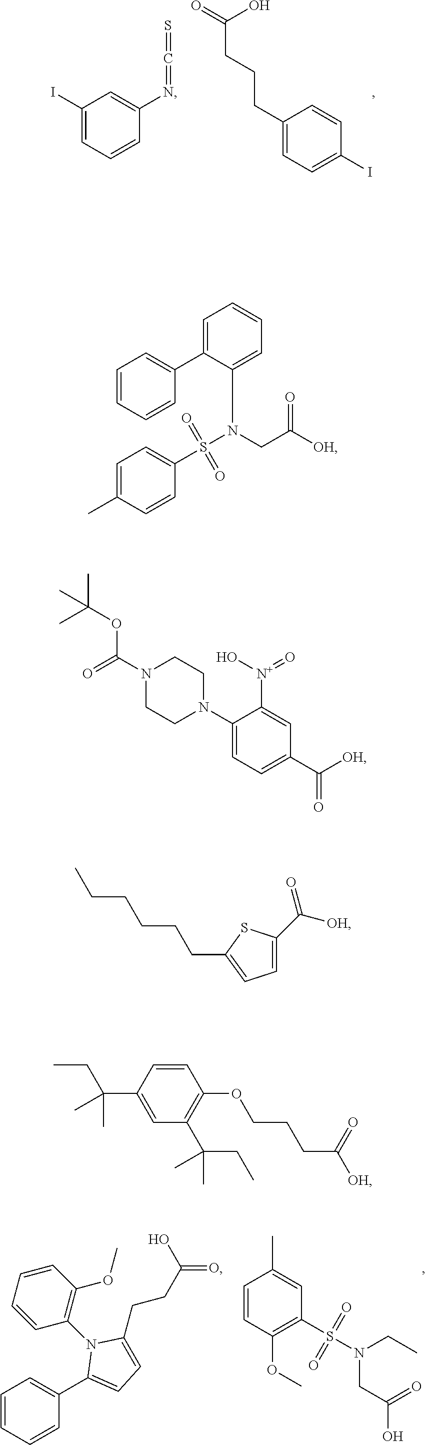

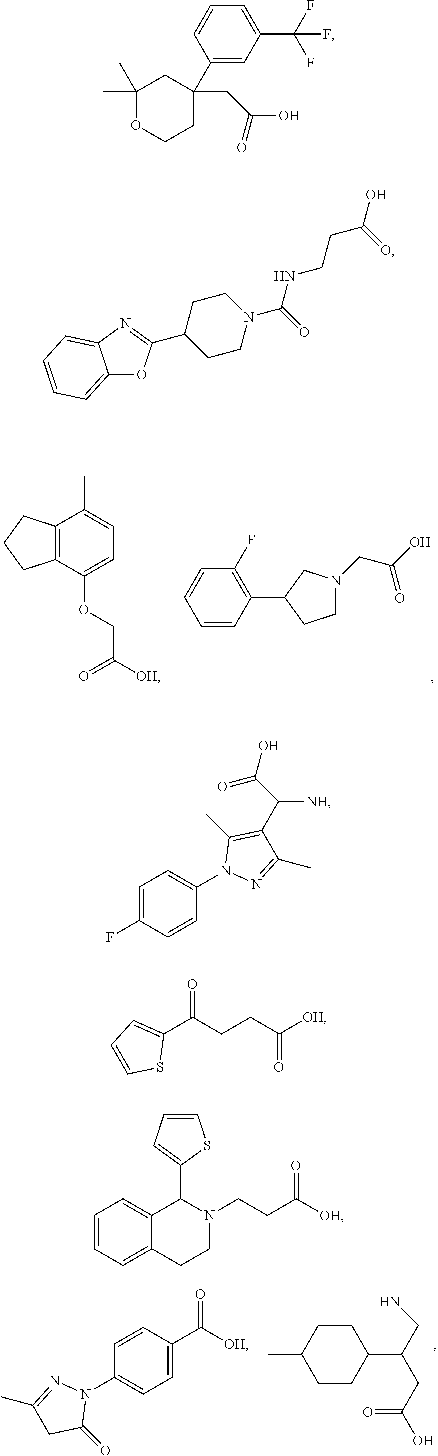

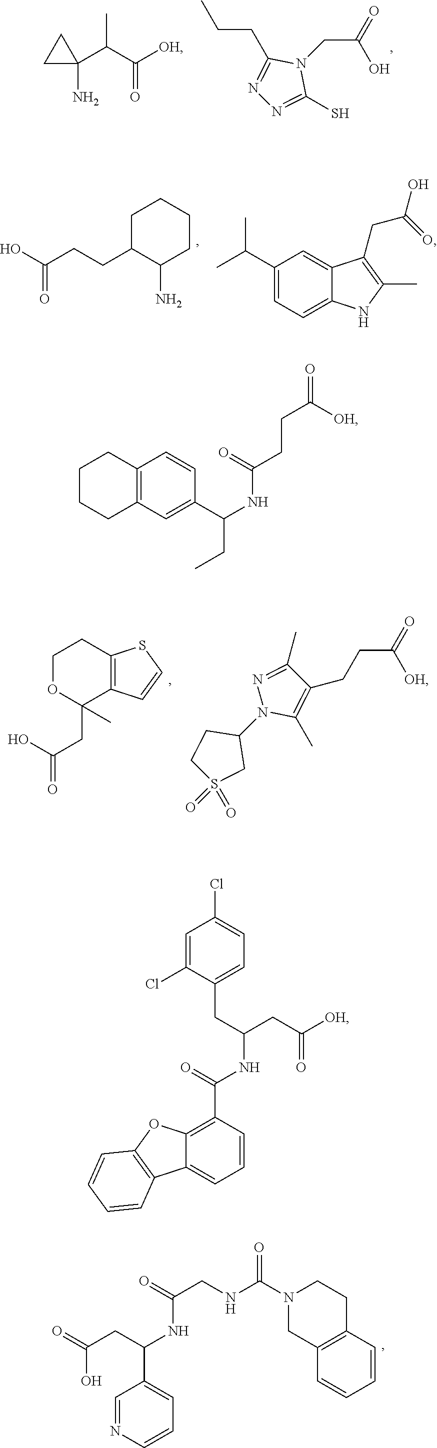

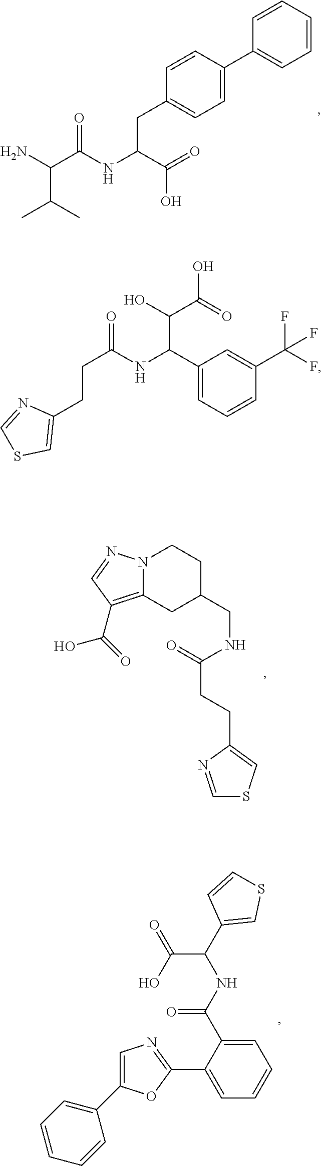 [SMILES: CC(C)C(N)C(=O)NC(Cc1ccc(-c2ccccc2)cc1)C(=O)O ; O=C(CCc1cscn1)NC(c1cccc(C(F)(F)F)c1)C(O)C(=O)O ; O=C(CCc1cscn1)NCC1CCn2ncc(C(=O)O)c2C1 ; O=C(NC(C(=O)O)c1ccsc1)c1ccccc1-c1ncc(-c2ccccc2)o1]

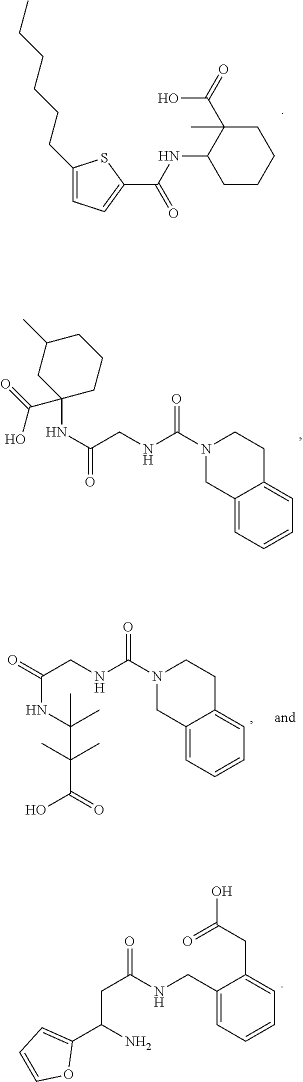 [SMILES: CCCCCCc1ccc(C(=O)NC2CCCCC2(C)C(=O)O)s1 ; CC1CCCC(NC(=O)CNC(=O)N2CCc3ccccc3C2)(C(=O)O)C1 ; CC(C)(NC(=O)CNC(=O)N1CCc2ccccc2C1)C(C)(C)C(=O)O ; NC(CC(=O)NCc1ccccc1CC(=O)O)c1ccco1]

8. The bivalent molecule of claim 1, wherein the small molecule is selected from:

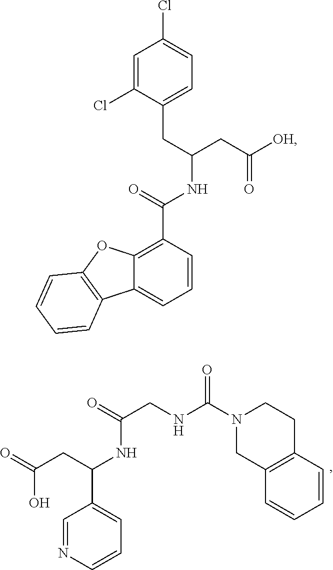 [SMILES: O=C(O)CC(Cc1ccc(Cl)cc1Cl)NC(=O)c1cccc2c1oc1ccccc12 ; O=C(O)CC(NC(=O)CNC(=O)N1CCc2ccccc2C1)c1cccnc1]

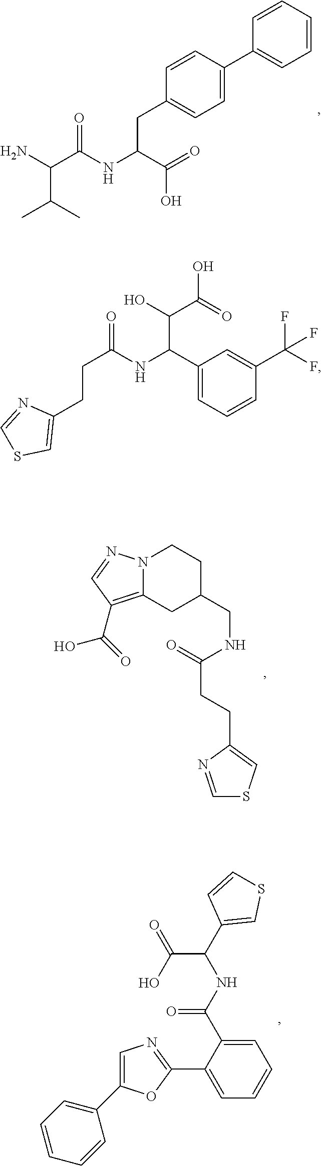 [SMILES: CC(C)C(N)C(=O)NC(Cc1ccc(-c2ccccc2)cc1)C(=O)O ; O=C(CCc1cscn1)NC(c1cccc(C(F)(F)F)c1)C(O)C(=O)O ; O=C(CCc1cscn1)NCC1CCn2ncc(C(=O)O)c2C1 ; O=C(NC(C(=O)O)c1ccsc1)c1ccccc1-c1ncc(-c2ccccc2)o1]

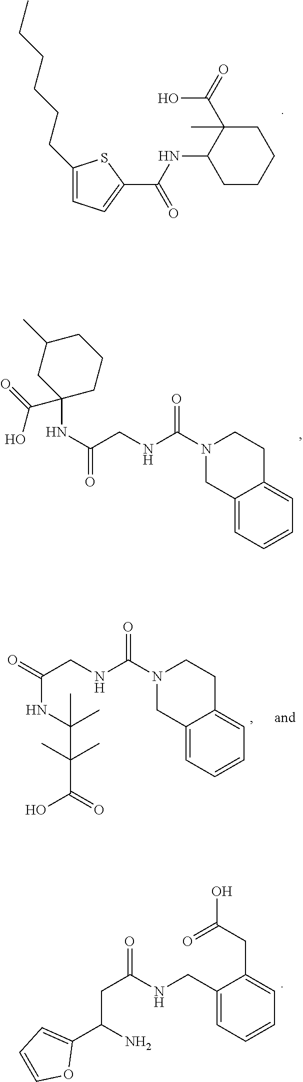 [SMILES: CCCCCCc1ccc(C(=O)NC2CCCCC2(C)C(=O)O)s1 ; CC1CCCC(NC(=O)CNC(=O)N2CCc3ccccc3C2)(C(=O)O)C1 ; CC(C)(NC(=O)CNC(=O)N1CCc2ccccc2C1)C(C)(C)C(=O)O ; NC(CC(=O)NCc1ccccc1CC(=O)O)c1ccco1]

9. The bivalent molecule of claim 1, wherein aberrant ubiquitination of the target to which the target binder binds causes a disease.

10. The bivalent molecule of claim 9, wherein the disease is an inherited ion channelopathy.

11. The bivalent molecule of claim 10, wherein the inherited ion channelopathy is selected from the group consisting of epilepsy, migraine, neuropathic pain, cardiac arrhythmias, long QT syndrome, Brugada syndrome, cystic fibrosis, diabetes, hyperinsulinemic hypoglycemia, Bartter syndrome, and diabetes insipidus.

12. The bivalent molecule of claim 10, wherein the disease is long QT syndrome.

13. The bivalent molecule of claim 10, wherein the disease is cystic fibrosis.

14. The bivalent molecule of claim 1, wherein the target to which the target binder binds is cystic fibrosis transmembrane conductance regulator (CFTR).

15. The bivalent molecule of claim 1, wherein the target binder is a small molecule.

16. The bivalent molecule of claim 15, wherein the small molecule binds to NBD1 domain of cystic fibrosis transmembrane conductance regulator (CFTR).

17. The bivalent molecule of claim 16, wherein the small molecule is selected from:

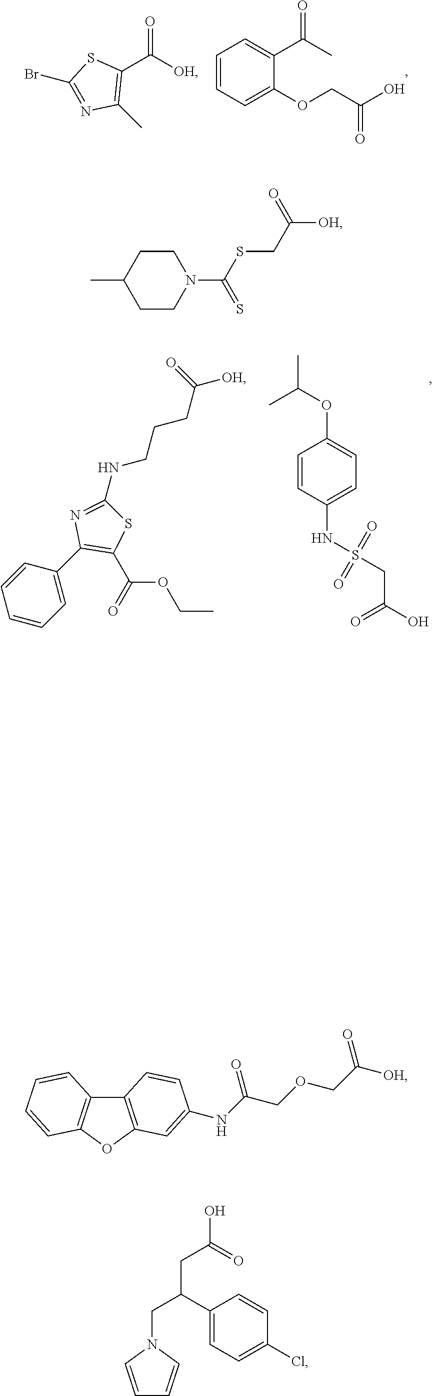

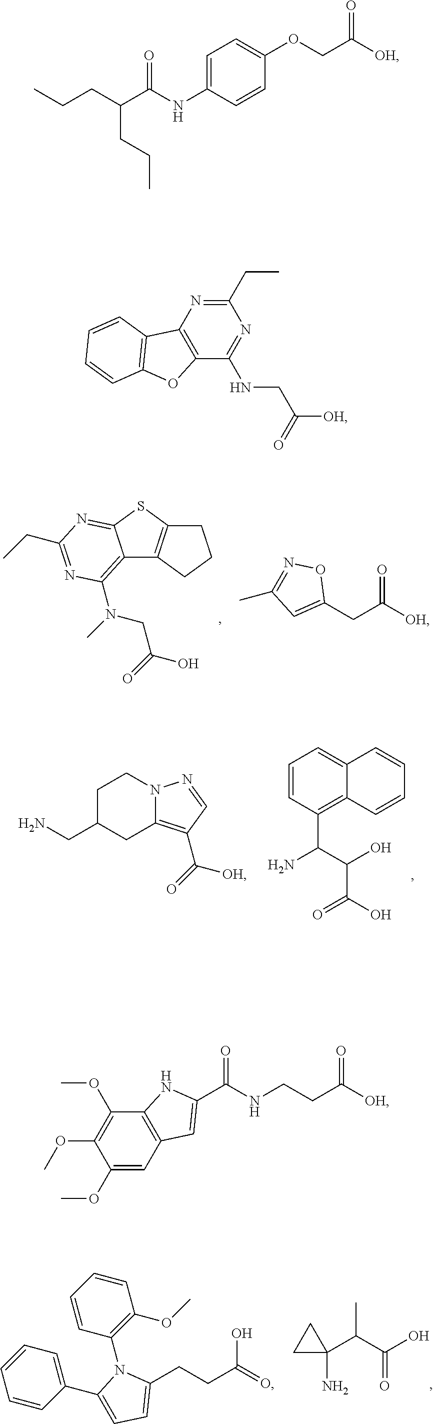

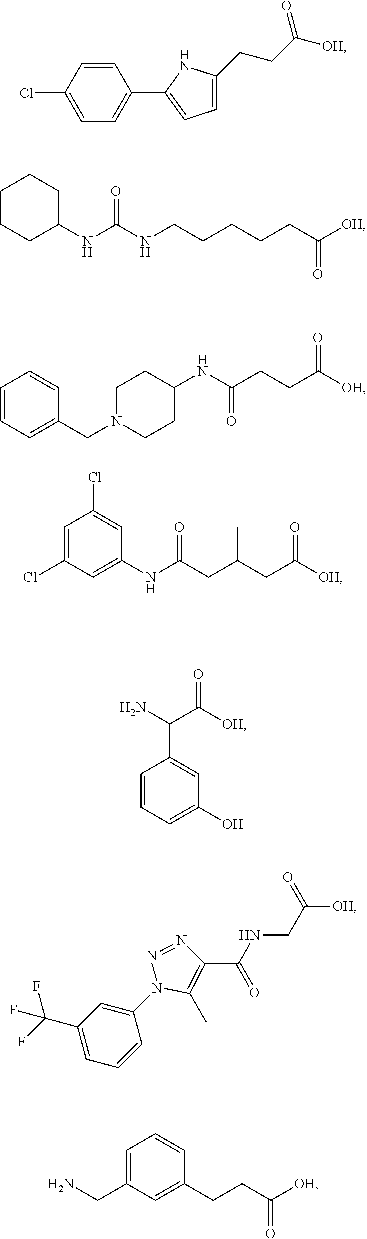

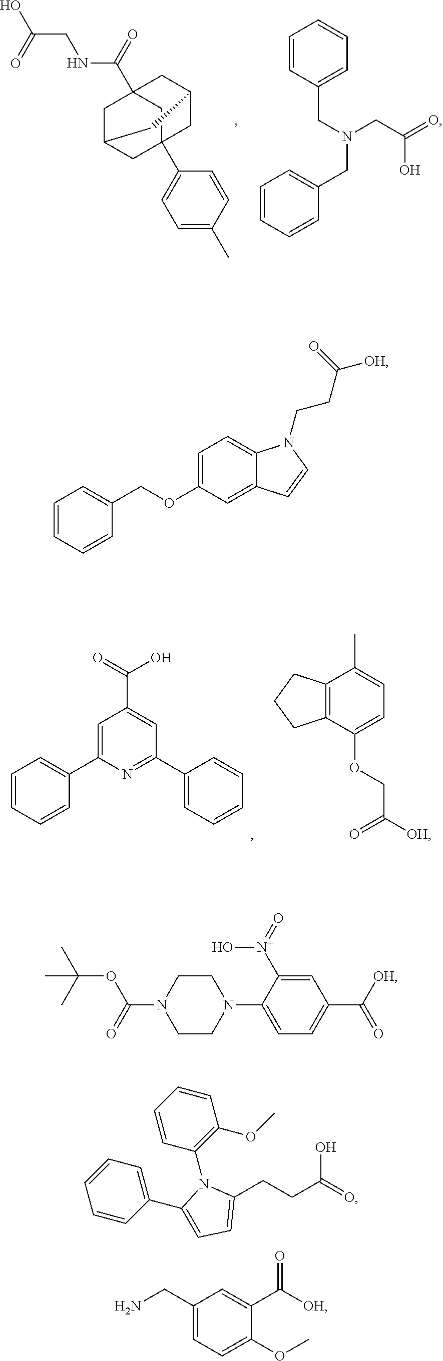

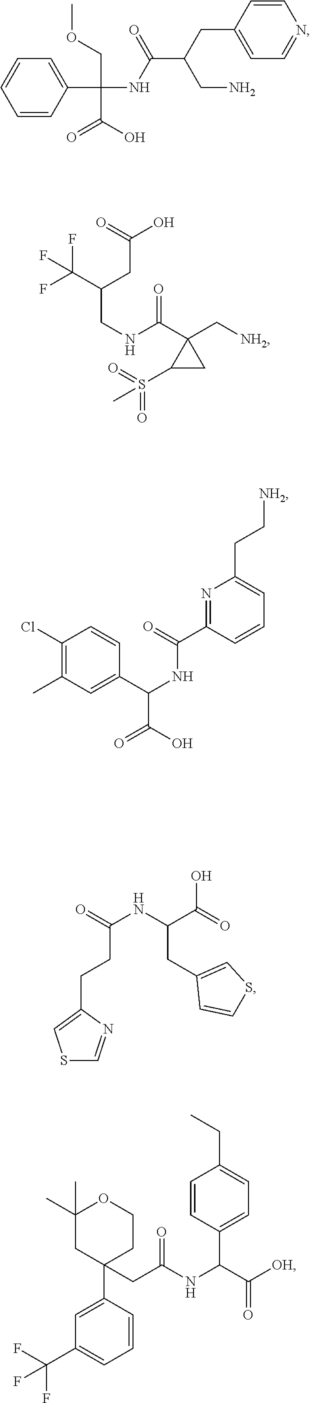 [SMILES: COCC(NC(=O)C(CN)Cc1ccncc1)(C(=O)O)c1ccccc1 ; CS(=O)(=O)C1CC1(CN)C(=O)NCC(CC(=O)O)C(F)(F)F ; Cc1cc(C(NC(=O)c2cccc(CCN)n2)C(=O)O)ccc1Cl ; O=C(CCc1cscn1)NC(Cc1ccsc1)C(=O)O ; CCc1ccc(C(NC(=O)CC2(c3cccc(C(F)(F)F)c3)CCOC(C)(C)C2)C(=O)O)cc1]

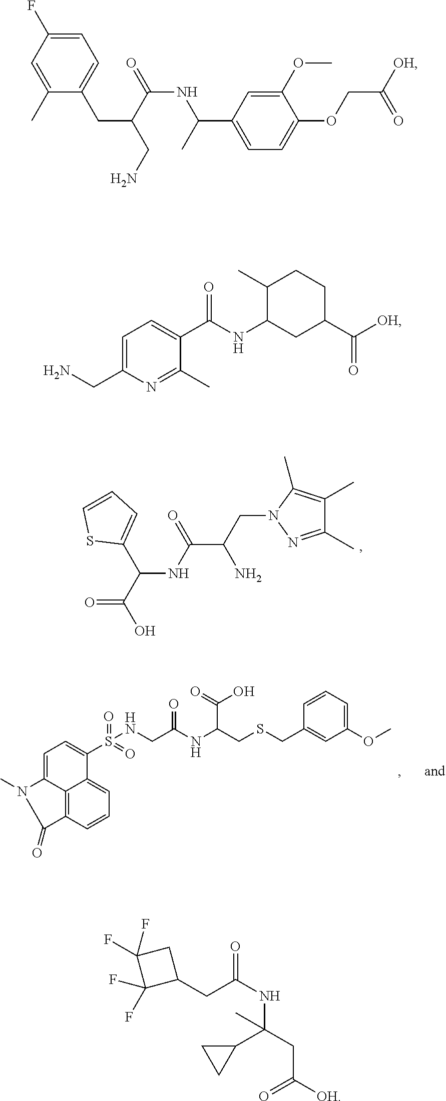

18. The bivalent molecule of claim 16, wherein the small molecule is selected from:

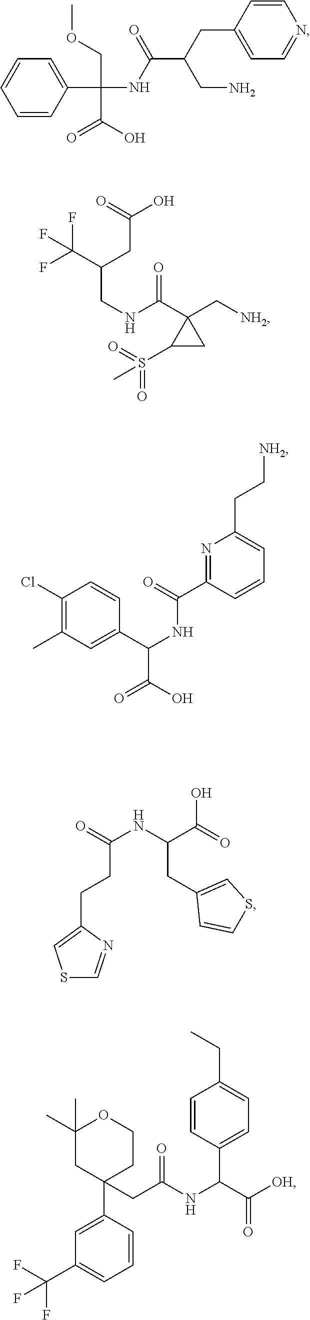 [SMILES: COCC(NC(=O)C(CN)Cc1ccncc1)(C(=O)O)c1ccccc1 ; CS(=O)(=O)C1CC1(CN)C(=O)NCC(CC(=O)O)C(F)(F)F ; Cc1cc(C(NC(=O)c2cccc(CCN)n2)C(=O)O)ccc1Cl ; O=C(CCc1cscn1)NC(Cc1ccsc1)C(=O)O ; CCc1ccc(C(NC(=O)CC2(c3cccc(C(F)(F)F)c3)CCOC(C)(C)C2)C(=O)O)cc1]

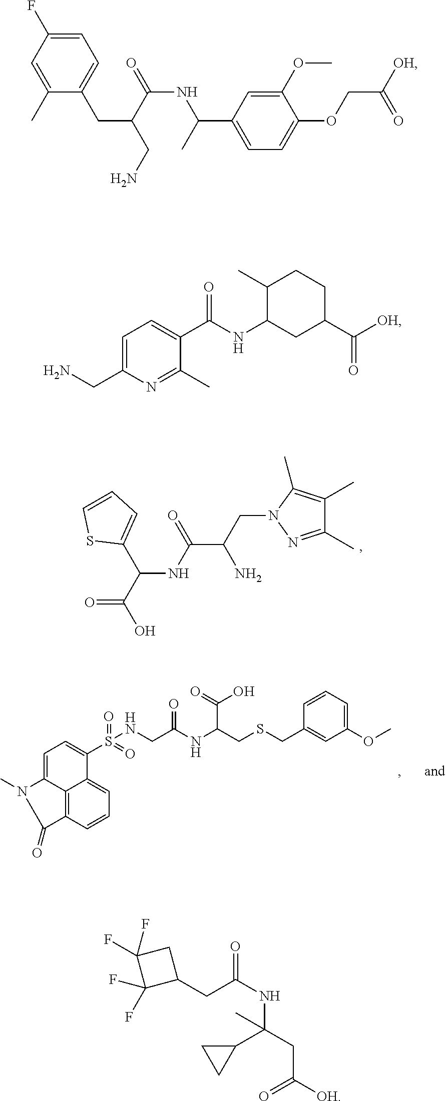

19. The bivalent molecule of claim 16, wherein the small molecule is selected from lumacaftor (VX-809), ivacaftor (VX-770), tezacaftor and elexacaftor.

20. The bivalent molecule of claim 1, wherein the linker is an alkyl, a polyethylene glycol (PEG), or a click linker.

21. A method of treating or ameliorating the effects of a disease in a subject, comprising administering to the subject an effective amount of a bivalent molecule of any one of the preceding claims.

22. The method of claim 21, wherein the subject is a human.

23. The method of claim 21, wherein the disease is selected from the group consisting of an inherited ion channelopathy, a cancer, a cardiovascular condition, an infectious disease, and a metabolic disease.

24. The method of claim 23, wherein the inherited ion channelopathy is selected from the group consisting of epilepsy, migraine, neuropathic pain, cardiac arrhythmias, long QT syndrome, Brugada syndrome, cystic fibrosis, diabetes, hyperinsulinemic hypoglycemia, Bartter syndrome, and diabetes insipidus.

25. The method of claim 23, wherein the inherited ion channelopathy is cystic fibrosis.

26. A method of identifying and preparing a small molecule binder targeting a protein of interest, comprising:
a) generating a DNA-encoded compound library;
b) incubating the library with the protein of interest;
c) washing off unbound molecules;
d) amplifying the oligonucleotide codes of the binding compounds by PCR and constructing an enriched compound library;
e) repeating steps b) to d) with the enriched library as necessary to further enrich the library containing the oligonucleotide codes of the binding compounds; and
f) identifying the small molecule binders by decoding the library generated in step
e) for binding validation.

27. The method of claim 26, wherein the protein of interest is cystic fibrosis transmembrane conductance regulator (CFTR).

28. The method of claim 26, wherein the protein of interest is a deubiquitinase (DUB).

29. The method of claim 26, wherein the DNA-encoded compound library is generated by a technique that is non-evolution-based or evolution-based.

30. The method of claim 26, wherein the decoding in step f) is carried out by Sanger sequencing, microarray, or high throughput sequencing.
